(19) United States
(12) Patent Application Publication
(10) Pub. No.: US 2011/0039785 A1
(43) Pub. Date: Feb. 17, 2011

(54) POLYPEPTIDE-NUCLEIC ACID CONJUGATES AND USES THEREOF

(75) Inventors: Richard Béliveau, Montreal (CA); Michel Demeule, Beaconsfield (CA); Christian Che, Longueuil (CA); Anthony Regina, Montreal (CA)

Correspondence Address: CLARK & ELBING LLP, 101 FEDERAL STREET, BOSTON, MA, 02110, US

(73) Assignee: Angiochem Inc., Montreal ,Quebec (CA)

(21) Appl. No.: 12/808,439

(22) PCT Filed: Dec. 19, 2008
(86) PCT No.: PCT/CA2008/002269

§ 371 Date: Nov. 1, 2010

Related U.S. Application Data
(60) Provisional application No. 61/008,825, filed on Dec. 20, 2007.

(51) Int. Cl. A61K 38/14; C07K 9/00; A61P 25/18; A61P 25/28; A61P 25/16; A61P 9/10; A61P 25/08; A61P 35/00; A61P 35/02

(52) U.S. Cl. 514/17.5; 530/322; 514/20.9; 514/17.9; 514/17.8; 514/17.7; 514/19.3; 514/19.8

(57) ABSTRACT

The present invention is directed to polypeptide-nucleic acid conjugates. These conjugates can allow for targeted application of a therapeutic RNAi agent across the blood-brain barrier to treat, for example, a cancer, neurodegenerative disease, or lysosomal storage disorder.

### FIELD OF THE INVENTION

The present invention relates to improvements in the field of drug delivery. More particularly, the invention relates to polypeptide-nucleic acid conjugates and their use for transporting a nucleic acid across the blood-brain barrier or into other tissues of a subject for the treatment of diseases such as cancer, neurodegenerative diseases, and lysosomal storage diseases.

### BACKGROUND OF THE INVENTION

In the development of a new therapy for brain pathologies, the blood-brain barrier (BBB) is considered as a major obstacle for the potential use of drugs for treating disorders of the central nervous system (CNS). The global market for CNS drugs was $33 billion in 1998, which was roughly half that of global market for cardiovascular drugs, even though in the United States, nearly twice as many people suffer from CNS disorders as from cardiovascular diseases. The reason for this imbalance is, in part, that more than 98% of all potential CNS drugs do not cross the blood-brain barrier. In addition, more than 99% of worldwide CNS drug development is devoted solely to CNS drug discovery, and less than 1% is directed to CNS drug delivery. This could explain why there is a lack of therapeutic options available for major neurological diseases.

The brain is shielded against potentially toxic substances by the presence of two barrier systems: the blood-brain barrier (BBB) and the blood-cerebrospinal fluid barrier (BCSFB). The BBB is considered to be the major route for the uptake of serum ligands since its surface area is approximately 5000-fold greater than that of BCSFB. The brain endothelium, which constitutes the BBB, represents the major obstacle for the use of potential drugs against many disorders of the CNS. As a general rule, only small lipophilic molecules may pass across the BBB, i.e., from circulating systemic blood to brain. Many drugs that have a larger size or higher hydrophobicity show promising results in animal studies for treating CNS disorders. Thus, peptide and protein therapeutics are generally excluded from transport from blood to brain, owing to the negligible permeability of the brain capillary endothelial wall to these drugs. Brain capillary endothelial cells (BCECs) are closely sealed by tight junctions, possess few fenestrae and few endocytic vesicles as compared to capillaries of other organs. BCECs are surrounded by extracellular matrix, astrocytes, pericytes, and microglial cells. The close association of endothelial cells with the astrocyte foot processes and the basement membrane of capillaries is important for the development and maintenance of the BBB properties that permit tight control of blood-brain exchange.

One method of treating diseases such as cancer, neurodegenerative diseases, or lysosomal storage diseases is gene silencing using RNA interference (RNAi). RNAi gene silencing can be accomplished using homologous short (21-23 bp) dsRNA fragments known as short interfering or “siRNA.” When a long dsRNA is introduced into a cell line, the cellular enzyme Dicer will cleave it into short interfering RNA (siRNA) molecules. This short interfering RNA molecule is now called the guided RNA. The guided RNA will guide the RNA-Induced-Silencing-Complex (RISC) to the homologous target mRNA. Once it forms a hybrid structure to the homologous mRNA sequence, the RISC will cleave the mRNA. As a result, protein that is encoded by the mRNA will no longer be produced, thereby causing the silencing of the gene.

RNA interference refers to the process of sequence-specific post-transcriptional gene silencing in animals mediated by short interfering RNAs (siRNAs). The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA. The presence of dsRNA in cells triggers the RNAi response through a mechanism that has yet to be fully characterized. This mechanism appears to be different from other known mechanisms involving double stranded RNA-specific ribonucleases, such as the interferon response that results from dsRNA-mediated activation of protein kinase PKR and 2′,5′-oligoadenylate synthetase resulting in non-specific cleavage of mRNA by ribonuclease L (see, for example, U.S. Pat. Nos. 6,107,094; 5,898,031; Clemens et al., J. Interferon & Cytokine Res., 17:503-524; 1997; Adah et al., Curr. Med. Chem. 8:1189, 2001).

### SUMMARY OF THE INVENTION

This invention features polypeptide-nucleic acid conjugates. These conjugates may be used for transporting RNAi agents, for example, siRNA agents, to cells, tissues, or organs to treat cancer, a neurodegenerative disease, or a lysosomal storage disease. The invention further features methods of synthesizing polypeptide-nucleic acid conjugates.

In one aspect, the invention features a polypeptide-nucleic acid conjugate. In a preferred embodiment, the polypeptide substantially identical to any of the sequences set forth in SEQ ID NOS:1-105 and 107-112 (e.g., AngioPep-1 (SEQ ID NO:67, AngioPep-2 (SEQ ID NO:97), AngioPep-3 (SEQ ID NO:107), AngioPep-4a (SEQ ID NO:108), AngioPep-4b (SEQ ID NO:109), AngioPep-5 (SEQ ID NO:110), AngioPep-6 (SEQ ID NO:111) and AngioPep-7 (SEQ ID NO:112)). The polypeptide may have the amino acid sequence set forth in SEQ ID NOS: 5, 8, 67, 75, 76, 77, 78, 79, 81, 82, 90, 91, or 97 (e.g., SEQ ID NOS:67 and 97). The conjugate may include a fragment of any of the polypeptides described herein (e.g., a fragment that is efficiently transported across the blood-brain barrier or is efficiently transported into particular cell types). The polypeptide-nucleic acid conjugate of the invention may be efficiently transported into a particular cell type (e.g., any one, two, three, four, or five of liver, lung, kidney, spleen, and muscle) or may cross the mammalian blood-brain barrier (BBB) efficiently (e.g., AngioPep-1, -2, -3, -4a, -4b, -5, and -6). In another embodiment, the conjugate is able to enter a particular cell type (e.g., any one, two, three, four, or five of liver, lung, kidney, spleen, and muscle) but does not cross the BBB efficiently (e.g., AngioPep-7). The polypeptide may be of any length, for example, at least 6, 7, 8, 9, 10, 11, 12, 13, 14, 15, 16, 17, 18, 19, 20, 21, 25, 35, 50, 75, 100, 200, or 500 amino acids. Preferably, the polypeptide is 10 to 50 amino acids in length. Likewise the nucleic acid may be any length (e.g., 15 to 25 nucleotides). The nucleic acid may be a DNA molecule, an RNA molecule, a modified nucleic acid (e.g., containing nucleotide analogs), or a combination thereof. The nucleic acid may be single-stranded, double-stranded, linear, circular (e.g., a plasmid), nicked circular, coiled, supercoiled, concatemerized, or charged. Additionally, nucleic acids may contain 5′ and 3′ sense and antisense strand terminal modifications and can have blunt or overhanging terminal nucleotides, or combinations thereof. The nucleic acid can be a short interfering RNA (siRNA), short hairpin RNA (shRNA), double-stranded RNA (dsRNA), or microRNA (miRNA) molecule. The siRNA, shRNA, dsRNA, and miRNA molecules of the invention can silence one of the following targets: vascular endothelial growth factor (VEGF), superoxide dismutase 1 (SOD-1), Huntingtin (Htt), α-secretase, β-secretase (BACE), γ-secretase, amyloid precursor protein (APP), sorting nexin-6 (SNX6), LINGO-1, Nogo-A, Nogo receptor 1 (NgR-1), and α-synuclein, and most preferably silence epidermal growth factor receptor (EGFR). In another embodiment, the siRNA, shRNA, dsRNA, or miRNA molecule of the invention has a nucleotide sequence with at least 70%, 80%, 90%, 95%, or 100% sequence identity, to any of the sequences set forth in SEQ ID NOS:117-119. The polypeptide-nucleic acid conjugates of the invention may be substantially pure. In another embodiment, the polypeptide is produced by recombinant genetic technology or chemical synthesis. The polypeptide-nucleic acid conjugates of the invention can be admixed or formulated with a pharmaceutically acceptable carrier.

In other embodiments, the conjugate includes a polypeptide including an amino acid sequence having the formula:

X1-X2-X3-X4-X5-X6-X7-X8-X9-X10-X11-X12-X13-X14-X15-X16-X17-X18-X19

where each of X1-X19 (e.g., X1-X6, X8, X9, X1′-X14, and X16-X19) is, independently, any amino acid (e.g., a naturally occurring amino acid such as Ala, Arg, Asn, Asp, Cys, Gln, Glu, Gly, H is, Ile, Leu, Lys, Met, Phe, Pro, Ser, Thr, Trp, Tyr, and Val) or absent and at least one of X1, X10, and X15 is arginine. In some embodiments, X7 is Ser or Cys; or X10 and X15 each are independently Arg or Lys. In some embodiments, the residues from X1 through X19, inclusive, are substantially identical to any of the amino acid sequences of any one of SEQ ID NOS:1-105 and 107-112 (e.g., AngioPep-1, AngioPep-2, AngioPep-3, AngioPep-4a, AngioPep-4b, AngioPep-5, AngioPep-6, and AngioPep-7). In some embodiments at least one (e.g., 2, 3, 4, or 5) of the amino acids X1-X19 are Arg (e.g., any one, two, or three of X1, X10, and X15).

Other exemplary polypeptides have a lysine or arginine at position 10, at position 15, or both (with respect to amino acid sequence of SEQ ID NO:1). The polypeptides of the invention may also have a serine or cysteine at position 7 (with respect to amino acid sequence of SEQ ID NO:1). Where multimerization of polypeptides is desired, the polypeptide may include a cysteine (e.g., at position 7).

In certain embodiments, the conjugate may include a polypeptide (e.g., any polypeptide described herein) that is modified (e.g., as described herein). The polypeptide may be amidated, acetylated, or both. Such modifications to polypeptides may be at the amino or carboxy terminus of said polypeptide. The conjugates of the invention also include peptidomimetics (e.g., those described herein) of any of the polypeptides described herein. The polypeptide may be in a multimeric form. For example, a polypeptide may be in a dimeric form (e.g., formed by disulfide bonding through cysteine residues).

The polypeptides of the invention may be efficiently transported into particular cells (e.g., liver, kidney, lung, muscle, or spleen cells) or may efficiently cross the BBB (e.g., SEQ ID NOS:5, 8, 67, 75, 76, 77, 78, 79, 81, 82, 90, 91, 107-111). In some embodiments, the polypeptide are efficiently transported into particular cells (e.g., liver, kidney, lung, muscle, or spleen cells) and are not efficiently transported across the BBB (e.g., AngioPep-7; SEQ ID NO:112). The polypeptide may be efficiently transported into at least one (e.g., at least two, three, four, or five) of a cell or tissue selected from the group consisting of liver, kidney, lung, muscle, or spleen.

For any of the polypeptides and conjugates described herein, the amino acid sequence may specifically exclude a polypeptide including or consisting of any of SEQ ID NOS:1-105 and 107-112 (e.g., any of SEQ ID NOs:1-96, AngioPep-1, AngioPep-2, AngioPep-3, AngioPep-4a, AngioPep-4b, AngioPep-5, AngioPep-6, and AngioPep-7). In some embodiments, the polypeptides and conjugates of the invention exclude the polypeptides of SEQ ID NOs:102, 103, 104 and 105. In other embodiments, the polypeptides and conjugates of the invention include these peptides.

In certain embodiments, a conjugate of the invention includes a polypeptide having an amino acid sequence described herein with at least one amino acid substitution (e.g., 2, 3, 4, 5, 6, 7, 8, 9, 10, 11, or 12 substitutions). In certain embodiments, the polypeptide may have an arginine at one, two, or three of the positions corresponding to positions 1, 10, and 15 of the amino acid sequence of any of SEQ ID NO:1, AngioPep-1, AngioPep-2, AngioPep-3, AngioPep-4a, AngioPep-4b, AngioPep-5, AngioPep-6, and AngioPep-7. For example, the polypeptide may contain 1 to 12 amino acid substitutions (e.g., SEQ ID NO:91). For example, the amino acid sequence may contain 1 to 10 (e.g., 9, 8, 7, 6, 5, 4, 3, 2) amino acid substitutions or 1 to 5 amino acid substitutions. In accordance with the invention, the amino acid substitution may be a conservative or non-conservative amino acid substitution.

In a second aspect, the invention features a method of treating (e.g., prophylactically) a subject having cancer by providing one or more polypeptide-nucleic acid conjugates of the invention to said subject in a therapeutically effective amount. In one embodiment, a polypeptide-nucleic acid conjugate is used to treat a cancer of the brain or central nervous system (e.g., where the polypeptide is efficiently transported across the BBB). In another embodiment, the cancer is a brain tumor, brain tumor metastasis, or a tumor that has metastasized. In other embodiments, a polypeptide-nucleic conjugate is used to treat a subject having a glioma, glioblastoma, hepatocellular carcinoma, lung cancer, or any of the cancers described herein.

In a third aspect, the invention features a method of treating (e.g., prophylactically) a subject having a neurodegenerative disease by providing one or more polypeptide-nucleic acid conjugates of the invention to said subject in a therapeutically effective amount. In one embodiment, the conjugate is used to treat a subject having multiple sclerosis, schizophrenia, epilepsy, Alzheimer's disease, Parkinson's disease, Huntington's disease, amyotrophic lateral sclerosis (ALS), a stroke, or any neurodegenerative disease described herein.

In a fourth aspect, the invention features a method of treating (e.g., prophylactically) a subject having a lysosomal storage disease by providing one or more polypeptide-nucleic acid conjugates of the invention to said subject in a therapeutically effective amount. In one embodiment, the conjugate is used to treat a subject having mucopolysaccharidosis (MPS-I; i.e., Hurler syndrome, Scheie syndrome), MPS-II (Hunter syndrome), MPS-IIIA (Sanfilippo syndrome A), MPS-IIIB (Sanfilippo syndrome B), (Sanfilippo syndrome C), (Sanfilippo syndrome D), MPS-VII (Sly syndrome), Gaucher's disease, Niemann-Pick disease, Fabry disease, Farber's disease, Wolman's disease, Tay-Sachs disease, Sandhoff disease, metachromatic leukodystrophy, Krabbé disease, or any of the lysosomal storage diseases described herein.

In a fifth aspect, the invention features a method of synthesizing a polypeptide-nucleic acid conjugate of the invention by conjugating a polypeptide described herein (e.g., an amino acid sequence substantially identical to any of the sequences of SEQ ID NOs:1-105 and 107-112) to a nucleic acid. In one embodiment, the polypeptide is conjugated to a nucleic acid with a covalent bond. In another embodiment, the polypeptide is conjugated to a nucleic acid with a disulfide bond. The polypeptide may be conjugated using a linker (e.g., any linker known in the art or described herein).

In any of the above aspects, the polypeptide-nucleic acid conjugate of the invention may be further conjugated to an agent (e.g., a therapeutic agent, detectable label, a protein, or a protein complex). Therapeutic agents include cytotoxic agents, alkylating agents, antibiotics, antineoplastic agents, antimetabolic agents, antiproliferative agents, tubulin inhibitors, topoisomerase I or II inhibitors, growth factors, hormonal agonists or antagonists, apoptotic agents, immunomodulators, and radioactive agents. Other cytotoxic agents include doxorubicin, methotrexate, camptothecin, homocamptothecin, thiocolchicine, colchicine, combretastatin, vinblastine, etoposide, cyclophosphamide, taxotere, melphalan, chlorambucil, combretastin A-4, podophyllotoxin, rhizoxin, rhizoxin-d, dolistatin, taxol, CC1065, ansamitocin p3, maytansinoid, and any combination thereof. Most preferably, the cytotoxic agent is paclitaxel. In another embodiment, the polypeptide-nucleic acid conjugate is conjugated to an antibody or antibody fragment.

By “blood-brain barrier” or “BBB” is meant a membranic structure that acts primarily to protect the brain from chemicals in the blood, while still allowing essential metabolic function. It is composed of endothelial cells, which are packed very tightly in brain capillaries. This higher density restricts passage of substances from the bloodstream much more than endothelial cells in capillaries elsewhere in the body.

The term “cancer” or “proliferative disease” is intended to mean any cellular proliferation whose unique trait is the loss of normal controls which results in unregulated growth, lack of differentiation, and/or ability to invade local tissues and metastasize. Cancer can develop in any tissue, in any organ, or in any cell type.

By “conjugate” is meant a combination of a vector and another compound or agent (e.g., an RNAi agent). The conjugation may be chemical in nature, such as via a linker, or genetic in nature for example by recombinant genetic technology, such as in a fusion protein with for example a reporter molecule (e.g., green fluorescent protein, β-galactosidase, or histamine tag).

By “double-stranded RNA” (dsRNA) is meant a double-stranded RNA molecule that can be used to silence a gene product via RNA interference.

By “fragment” is meant a polypeptide originating from a portion of an original or parent sequence or from an analog of said parent sequence. Fragments encompass polypeptides having truncations of one or more (e.g., 2, 3, 4, 5, 6, 7, 8, 9, 10, 11, 12, 13, 14, 15, 16, 17, 18, or 19) amino acids wherein the truncation may originate from the amino terminus (N-terminus), carboxy terminus (C-terminus), or from the interior of the protein. A fragment may comprise the same sequence as the corresponding portion of the original sequence. Biologically active fragments of the vector (i.e., polypeptide) described herein are encompassed by the present invention.

By “lysosomal storage disease” is meant any disorder that results from defects in lysosomal function. Exemplary lysosomal storage diseases include the mucopolysaccharidoses (MPS, e.g., Hunter syndrome), leukodystrophies (e.g., metachromatic leukodystrophy), gangliosidoses (e.g., Tay-Sachs disease), mucolipidoses, lipidoses (e.g., Gaucher's disease), and glycoproteinoses. Other lysosomal storage diseases are described herein.

By “microRNA” (miRNA) is meant a single-stranded RNA molecule that can be used to silence a gene product via RNA interference.

By “modulate” is meant that the expression of a gene, or level of an RNA molecule or equivalent RNA molecules encoding one or more proteins or protein subunits, or activity of one or more proteins or protein subunits is up-regulated or down-regulated, such that expression, level, or activity is greater than or less than that observed in the absence of the modulator. For example, the term modulate can include inhibition.

By “neurodegenerative disease” is meant any disease or condition affecting the mammalian brain, central nervous system (CNS), the peripheral nervous system, or the autonomous nervous system wherein neurons are lost or deteriorate. Exemplary neurodegenerative diseases include Alzheimer's disease, Parkinson's disease, Krabbé disease, multiple sclerosis, narcolepsy, and HIV-associated dementia.

A “non-naturally occurring amino acid” is an amino acid that is not naturally produced or found in a mammal.

By “subject” is meant any human or non-human animal (e.g., a mammal). Other animals that can be treated using the methods and compositions of the invention include horses, dogs, cats, pigs, goats, rabbits, hamsters, monkeys, guinea pigs, rats, mice, lizards, snakes, sheep, cattle, fish, and birds.

By “pharmaceutically acceptable carrier” is meant a carrier physiologically acceptable to a patient while retaining the therapeutic properties of the compound with which it is administered.

By “providing” is meant, in the context of a conjugate of the invention, to bring the conjugate into contact with a target cell or tissue either in vivo or in vitro. A vector or conjugate may be provided by administering the vector or conjugate to a subject.

By “RNAi agent” is meant any agent or compound that exerts a gene silencing effect by way of an RNA interference pathway. RNAi agents include any nucleic acid molecules that are capable of mediating sequence-specific RNAi, for example, a short interfering RNA (siRNA), double-stranded RNA (dsRNA), microRNA (miRNA), short hairpin RNA (shRNA), short interfering oligonucleotide, short interfering nucleic acid, short interfering modified oligonucleotide, chemically-modified siRNA, and post-transcriptional gene silencing RNA (ptgsRNA).

By “silencing” or “gene silencing” is meant that the expression of the gene, or level of RNA molecules or equivalent RNA molecules encoding one or more proteins or protein subunits, or activity of one or more proteins or protein subunits, is reduced in the presence of an RNAi agent below that observed in the absence of the RNAi agent (e.g., an siRNA). In one embodiment, gene silencing with a siRNA molecule reduces a gene product expression below the level observed in the presence of an inactive or attenuated molecule, or below that level observed in the presence of, for example, a siRNA molecule with scrambled sequence or with mismatches.

By “short hairpin RNA” or “shRNA” is meant a sequence of RNA that makes a tight hairpin turn that can be used to silence a gene product via RNA interference.

By “small inhibitory RNA,” “short interfering RNA,” or “siRNA” is meant a class of 10-40 (e.g., 15-25, such as 21) nucleotide-long double-stranded RNA molecules. Most notably, siRNA are typically involved in the RNA interference (RNAi) pathway by which the siRNA interferes with the expression of a specific gene product (e.g., EGFR).

By “substantial identity” or “substantially identical” is meant a polypeptide or polynucleotide sequence that has the same polypeptide or polynucleotide sequence, respectively, as a reference sequence, or has a specified percentage of amino acid residues or nucleotides, respectively, that are the same at the corresponding location within a reference sequence when the two sequences are optimally aligned. For example, an amino acid sequence that is “substantially identical” to a reference sequence has at least 50%, 60%, 70%, 75%, 80%, 85%, 90%, 95%, 96%, 97%, 98%, 99%, or 100% identity to the reference amino acid sequence. For polypeptides, the length of comparison sequences will generally be at least 5, 6, 7, 8, 9, 10, 11, 12, 13, 14, 15, 16, 17, 18, 19, or 20 contiguous amino acids, more preferably at least 25, 50, 75, 90, 100, 150, 200, 250, 300, or 350 contiguous amino acids, and most preferably the full-length amino acid sequence. For nucleic acids, the length of comparison sequences will generally be at least 5 contiguous nucleotides, preferably at least 10, 11, 12, 13, 14, 15, 16, 17, 18, 19, 20, 21, 22, 23, 24, or 25 contiguous nucleotides, and most preferably the full-length nucleotide sequence. Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

By “substantially pure” or “isolated” is meant a compound (e.g., a polypeptide or conjugate) that has been separated from other chemical components. Typically, the compound is substantially pure when it is at least 30%, by weight, free from other components. In certain embodiments, the preparation is at least 50%, 60%, 75%, 85%, 90%, 95%, 96%, 97%, 98%, or 99% by weight, free from other components. A purified polypeptide may be obtained, for example, by expression of a recombinant polynucleotide encoding such a polypeptide or by chemically synthesizing the polypeptide. Purity can be measured by any appropriate method, for example, column chromatography, polyacrylamide gel electrophoresis, or by HPLC analysis.

By “sense region” is meant a nucleotide sequence of a nucleic acid of the invention having complementarity to an antisense region of another nucleic acid. In addition, the sense region of a nucleic acid of the invention can include a nucleotide sequence having homology with a target gene nucleotide sequence. By “antisense region” is meant a nucleotide sequence of a nucleic acid of the invention having complementarity to a target gene nucleotide sequence.

By “target nucleic acid” is meant any nucleic acid sequence whose expression or activity is to be modulated. The target nucleic acid can be DNA or RNA.

By “agent” is meant any compound, for example, an antibody, or a therapeutic agent, a detectable label (e.g., a marker, tracer, or imaging compound).

By “therapeutic agent” is meant any compound having a biological activity. Therapeutic agents encompass the full spectrum of treatments for a disease or disorder. A therapeutic agent may act in a manner that is prophylactic or preventive, including those that incorporate procedures designed to target individuals that can be identified as being at risk (pharmacogenetics); or in a manner that is ameliorative or curative in nature; or may act to slow the rate or extent of the progression of a disease or disorder; or may act to minimize the time required, the occurrence or extent of any discomfort or pain, or physical limitations associated with recuperation from a disease, disorder or physical trauma; or may be used as an adjuvant to other therapies and treatments.

By “treatment,” “treating,” and the like are meant obtaining a desired pharmacologic and/or physiologic effect, e.g., inhibition of cancer cell growth, death of a cancer cell or amelioration of a neurodegenerative or lysosomal storage disease. Treament includes inhibiting a disease, (e.g., arresting its development) and relieving a disease (e.g., reducing symptoms associated with a disease). Treatment as used herein covers any administration of a pharmaceutical agent or compound to an individual to treat, cure, alleviate, improve, diminish, or inhibit a condition in the individual, including, administering a carrier-agent conjugate to an individual. By “treating cancer,” “preventing cancer,” or “inhibiting cancer” is meant causing a reduction in the size of a tumor or the number of cancer cells, slowing or inhibiting an increase in the size of a tumor or cancer cell proliferation, increasing the disease-free survival time between the disappearance of a tumor or other cancer and its reappearance, preventing or reducing the likelihood of an initial or subsequent occurrence of a tumor or other cancer, or reducing an adverse symptom associated with a tumor or other cancer. In a desired embodiment, the percent of tumor or cancerous cells surviving the treatment is at least 20, 40, 60, 80, or 100% lower than the initial number of tumor or cancerous cells, as measured using any standard assay. Desirably, the decrease in the number of tumor or cancerous cells induced by administration of a compound of the invention is at least 2, 5, 10, 20, or 50-fold greater than the decrease in the number of non-tumor or non-cancerous cells. Desirably, the methods of the present invention result in a decrease of 20, 40, 60, 80, or 100% in the size of a tumor or number of cancerous cells as determined using standard methods. Desirably, at least 20, 40, 60, 80, 90, or 95% of the treated subjects have a complete remission in which all evidence of the tumor or cancer disappears. Desirably, the tumor or cancer does not reappear or reappears after no less than 5, 10, 15, or 20 years.

By “treating prophylactically” is meant reducing the frequency of occurrence of a disease or the severity of the disease by administering an agent prior to appearance of a symptom of that disease. The prophylactic treatment may completely prevent or reduce appears of the disease or a symptom thereof and/or may be therapeutic in terms of a partial or complete cure for a disease and/or adverse effect attributable to the disease. Prophylactic treatment may include reducing or preventing a disease or condition (e.g., preventing cancer) from occurring in an individual who may be predisposed to the disease but has not yet been diagnosed as having it.

By “vector” is meant a compound or molecule such as a polypeptide that is able to transport another compound. For example, transport (e.g., of an RNAi agent) may occur across the blood-brain barrier or to a specific tissue or organ (e.g., the liver, lungs, kidney, spleen, or muscle) using a vector. The vector may bind to receptors present on brain endothelial cells and thereby be transported across the blood-brain barrier by transcytosis. The vector may be a molecule for which high levels of transendothelial transport may be obtained, without affecting the blood-brain barrier integrity. The vector may be a protein, a peptide, or a peptidomimetic and may be naturally occurring or produced by chemical synthesis or recombinant genetic technology (genetic engineering).

By a vector that is “efficiently transported across the BBB” is meant a vector that is able to cross the BBB at least as efficiently as AngioPep-6 (i.e., greater than 38.5% that of AngioPep-1 (250 nM) in the in situ brain perfusion assay described in U.S. application Ser. No. 11/807,597, filed May 29, 2007, hereby incorporated by reference). Accordingly, a vector or conjugate that is “not efficiently transported across the BBB” is transported to the brain at lower levels (e.g., transported less efficiently than AngioPep-6).

By a vector or conjugate which is “efficiently transported to a particular cell type” is meant a vector or conjugate that is able to accumulate (e.g., either due to increased transport into the cell, decreased efflux from the cell, or a combination thereof) in that cell type at least 10% (e.g., 25%, 50%, 100%, 200%, 500%, 1,000%, 5,000%, or 10,000%) greater extent than either a control substance, or, in the case of a conjugate, as compared to the unconjugated agent. Such activities are described in detail in PCT Publication No. WO 2007/009229, hereby incorporated by reference.

If a “range” or “group of substances” is mentioned with respect to a particular characteristic (e.g., temperature, concentration, time and the like), the invention relates to and explicitly incorporates herein each and every specific member and combination of sub-ranges or sub-groups therein. Thus, for example, with respect to a length of from 9 to 18 amino acids, is to be understood as specifically incorporating herein each and every individual length, e.g., a length of 18, 17, 15, 10, 9, and any number therebetween. Therefore, unless specifically mentioned, every range mentioned herein is to be understood as being inclusive. For example, in the expression from 5 to 19 amino acids long is to be as including 5 and 19. This similarly applies with respect to other parameters such as sequences, length, concentrations, elements, and the like.

The sequences, regions, and portions defined herein each include each and every individual sequence, region, and portion described thereby as well as each and every possible sub-sequence, sub-region, and sub-portion whether such sub-sequences, sub-regions, and sub-portions are defined as positively including particular possibilities, as excluding particular possibilities or a combination thereof. For example, an exclusionary definition for a region may read as follows: “provided that said polypeptide is no shorter than 4, 5, 6, 7, 8 or 9 amino acids. A further example of a negative limitation is the following; a sequence including SEQ ID NO:X with the exclusion of a polypeptide of SEQ ID NO:Y; etc. An additional example of a negative limitation is the following; provided that said polypeptide is not (does not include or consist of) SEQ ID NO:Z.

### BRIEF DESCRIPTION OF THE DRAWINGS

FIG. 1 is a schematic diagram showing the mechanism of inhibition by RNA interference (RNAi).

FIG. 2 is a schematic diagram showing the conjugation of AngioPep-2 (SEQ ID NO:97) to a siRNA molecule with the cross-linker sulfo-LC-SPDP. The use of this cross-linker results in the cleavable disulfide bond between the siRNA molecule and AngioPep-2.

FIG. 3 is a drawing of the cross-linker sulfo-LC-SPDP. This cross-linker can be used to join the polypeptide and RNAi agents of the invention by creating a cleavable disulfide bond.

FIG. 4 is a schematic diagram showing exemplary cleavable and noncleavable Angiopep-2-siRNA conjugates, where Angiopep-2 is conjugated to the sense strand of the siRNA.

FIG. 5 is a set of graphs showing siRNA activity of a cleavable siRNA conjugate, a non-cleavable siRNA conjugate, and a control (unconjugated siRNA).

FIG. 6 is a graph showing uptake of cleavable and non-cleavable siRNA conjugates.

FIG. 7 is a schematic diagram of modified forms of Angiopep-2; Cys-Angiopep-2 (SEQ ID NO:113) and 6-maleimidohexanoic acid (6-MHA)-derivitized Angiopep-2 are shown.

FIG. 8 is a schematic diagram showing the reaction of an exemplary derivatized RNA molecule with the reducing agent tris(2-carboxyethyl) phosphine (TCEP) to a free thiol, followed by further reaction with 2,2′-dipyridyl disulfide (Py-S-S-Py) to form an activated siRNA.

FIGS. 9A-9C show HPLC traces of the siRNA with a free thio (FIG. 9A), synthesis of the activated siRNA (FIG. 9B), and Cys-Angiopep-2 (FIG. 9C).

FIG. 10 is a schematic diagram showing the conjugation reaction of activated siRNA with Cys-Angiopep-2.

FIGS. 11A-11C are graphs showing HPLC traces and relative retention times of the activated siRNA (FIG. 11A), Cys-Angiopep-2 (FIG. 11B), and the siRNA conjugate (FIG. 11C).

FIG. 12 is a graph showing results of mass spectroscopy performed on the siRNA conjugate.

FIG. 13 is a schematic diagram showing the conjugation reaction of siRNA with a free thiol and Angiopep-2 derivatized with a maleimide.

FIGS. 14A-14C are graphs showing HPLC traces and relative retention times of the siRNA with a free thiol (FIG. 14A), the Angiopep-2-maleimide (FIG. 14B), and the siRNA+polypeptide crude reaction mixture (FIG. 14C).

FIGS. 15A-15B are graphs showing an HPLC trace of the purified siRNA-polypeptide conjugate (FIG. 15A) and results of mass spectroscopy performed on the conjugate (FIG. 15B).

FIG. 16 is a schematic diagram showing structure of an antisense strand siRNA conjugated to the fluorescent label Alexa 488.

FIGS. 17A-17B are graphs showing HPLC traces of additional cleavable (FIG. 17A) and non-cleavable (FIG. 17B) Angiopep-2 conjugates. Also shown are the unconjugated Angiopep-2 peptides and a control siRNA.

FIG. 18 is a graph showing HPLC traces of fluorescently labeled siRNA-Angiopep-2 conjugates, both cleavable and non-cleavable.

FIGS. 19A-19B are a set of graphs showing HPLC traces of cleavable (FIG. 19A) and non-cleavable (FIG. 19B) siRNA conjugates before and following the iodination procedure described herein.

FIG. 20 is a graph showing the results of an in situ brain perfusion assay performed in mice using the radiolabeled siRNA conjugates. Inulin is shown as a control.

FIG. 21 is a graph showing results from an in situ perfusion assay performed in mice using radiolabeled siRNA conjugates. Amounts of radiolabeled siRNA conjugates in total brain, parenchyma, and brain capillaries was measured. Inulin was used as a control.

FIG. 22 is a graph showing results from an in situ perfusion assay using fluorescently labeled siRNA conjugates. Alex-488 and an unlabeled siRNA are used as controls.

FIG. 23 is a graph showing results from an in vitro blood-brain barrier model using cleavable and non-cleavable siRNA conjugates. Holo-transferrin was used as a control.

FIG. 24 is a graph showing saturatable transport of the radiolabeled siRNA conjugates in the in vitro BBB model.

FIG. 25 is a graph showing transport of fluorescently labeled siRNA conjugates in the in vitro BBB model. Unconjugated fluorescently labeled siRNA was used as a control.

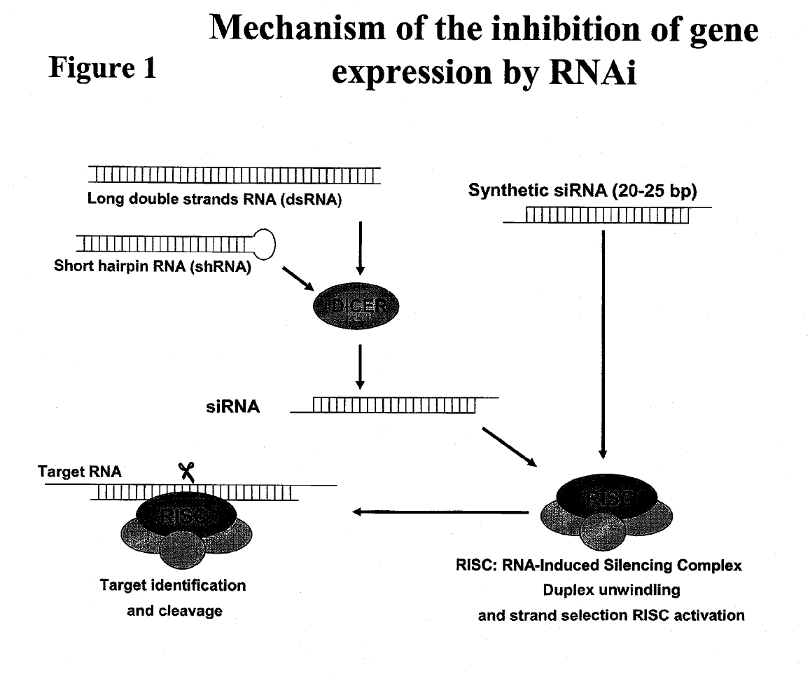

### DETAILED DESCRIPTION OF THE INVENTION

The present invention relates to conjugates of the polypeptides that can act as vectors to transport an RNA interference (RNAi) agent to the brain, central nervous system (CNS), or other organs. Different modes of RNAi, such as siRNA, shRNA, dsRNA, and miRNA, are useful for the silencing of specific cellular genes for the treatment of cancer, neurodegenerative diseases, lysosomal storage diseases, and other conditions. In addition to transporting the RNAi agent, the polypeptide component of the conjugates can stabilize, protect (e.g., nuclease protection), or target the RNAi therapeutic agent to specific cells, tissues, or organs of the treated individual. In addition, other agents that are unable or ineffective at crossing the blood-brain barrier by themselves, can be transported across the blood-brain barrier when attached or coupled to these polypeptide-nucleic acid conjugates. In other cases, an agent that is able to cross the blood-brain barrier may see its transport increase when conjugated to the polypeptide vectors described herein. Such conjugates can be in the form of a composition, such as a pharmaceutical composition, for treatment or diagnosis of a condition or disease.

### Polypeptide Vectors

The compounds, conjugates, and compositions of the invention features any of polypeptides described herein, for example, any of the peptides described in Table 1 (e.g., a peptide defined in any of SEQ ID NOS:1-105 and 107-112 such as AngioPep-1 or AngioPep-2), or any fragment, analog, derivative, or variant thereof. In certain embodiments, the polypeptide may have at least 35%, 40%, 50%, 60%, 70%, 80%, 90%, 95%, 99%, or even 100% identity to a polypeptide described herein. The polypeptide may have one or more (e.g., 2, 3, 4, 5, 6, 7, 8, 9, 10, 11, 12, 13, 14, or 15) substitutions relative to one of the sequences described herein. Other modifications are described in greater detail below.

The invention also features fragments of these polypeptides (e.g., a functional fragment). In certain embodiments, the fragments are capable of efficiently being transported to or accumulating in a particular cell type (e.g., liver, eye, lung, kidney, or spleen) or are efficiently transported across the BBB. Truncations of the polypeptide may be 1, 2, 3, 4, 5, 6, 7, 8, 9, 10, 11, 12, or more amino acids from either the N-terminus of the polypeptide, the C-terminus of the polypeptide, or a combination thereof. Other fragments include sequences where internal portions of the polypeptide are deleted.

Additional polypeptides may be identified using one of the assays or methods described herein. For example, a candidate vector may be produced by conventional peptide synthesis, conjugated with paclitaxel and administered to a laboratory animal. A biologically-active vector may be identified, for example, based on its efficacy to increase survival of an animal injected with tumor cells and treated with the conjugate as compared to a control which has not been treated with a conjugate (e.g., treated with the unconjugated agent). For example, a biologically active polypeptide may be identified based on its location in the parenchyma in an in situ cerebral perfusion assay.

Assays to determine accumulation in other tissues may be performed as well. Labeled conjugates of a polypeptide can be administered to an animal, and accumulation in different organs can be measured. For example, a polypeptide conjugated to a detectable label (e.g., a near-IR fluorescence spectroscopy label such as Cy5.5) allows live in vivo visualization. Such a polypeptide can be administered to an animal, and the presence of the polypeptide in an organ can be detected, thus allowing determination of the rate and amount of accumulation of the polypeptide in the desired organ. In other embodiments, the polypeptide can be labeled with a radioactive isotope (e.g., ¹²⁵I). The polypeptide is then administered to an animal. After a period of time, the animal is sacrificed and the organs are extracted. The amount of radioisotope in each organ can then be measured using any means known in the art. By comparing the amount of a labeled candidate polypeptide in a particular organ relative to the amount of a labeled control polypeptide, the ability of the candidate polypeptide to access and accumulate in a particular tissue can be ascertained. Appropriate negative controls include any peptide or polypeptide known not to be efficiently transported into a particular cell type.

TABLE 1
Exemplary Polypeptides

| SEQ ID | |
|---|---|
| NO: | |
| 1 | T F V Y G G C R A K R N N F K S A E D |
| 2 | T F Q Y G G C M G N G N N F V T E K E |
| 3 | P F F Y G G C G G N R N N F D T E E Y |
| 4 | S F Y Y G G C L G N K N N Y L R E E E |
| 5 | T F F Y G G C R A K R N N F K R A K Y |
| 6 | T F F Y G G C R G K R N N F K R A K Y |
| 7 | T F F Y G G C R A K K N N Y K R A K Y |
| 8 | T F F Y G G C R G K K N N F K R A K Y |
| 9 | T F Q Y G G C R A K R N N F K R A K Y |
| 10 | T F Q Y G G C R G K K N N F K R A K Y |
| 11 | T F F Y G G C L G K R N N F K R A K Y |
| 12 | T F F Y G G S L G K R N N F K R A K Y |
| 13 | P F F Y G G C G G K K N N F K R A K Y |
| 14 | T F F Y G G C R G K G N N Y K R A K Y |
| 15 | P F F Y G G C R G K R N N F L R A K Y |
| 16 | T F F Y G G C R G K R N N F K R E K Y |
| 17 | P F F Y G G C R A K K N N F K R A K E |
| 18 | T F F Y G G C R G K R N N F K R A K D |
| 19 | T F F Y G G C R A K R N N F D R A K Y |
| 20 | T F F Y G G C R G K K N N F K R A E Y |
| 21 | P F F Y G G C G A N R N N F K R A K Y |
| 22 | T F F Y G G C G G K K N N F K T A K Y |
| 23 | T F F Y G G C R G N R N N F L R A K Y |
| 24 | T F F Y G G C R G N R N N F K T A K Y |
| 25 | T F F Y G G S R G N R N N F K T A K Y |
| 26 | T F F Y G G C L G N G N N F K R A K Y |
| 27 | T F F Y G G C L G N R N N F L R A K Y |
| 28 | T F F Y G G C L G N R N N F K T A K Y |
| 29 | T F F Y G G C R G N G N N F K S A K Y |
| 30 | T F F Y G G C R G K K N N F D R E K Y |
| 31 | T F F Y G G C R G K R N N F L R E K E |
| 32 | T F F Y Q G C R G K G N N F D R A K Y |
| 33 | T F F Y G G S R G K G N N F D R A K Y |
| 34 | T F F Y G G C R G N G N N F V T A K Y |
| 35 | P F F Y G G C G G K G N N Y V T A K Y |
| 36 | T F F Y G G C L G K G N N F L T A K Y |
| 37 | S F F Y G G C L G N K N N F L T A K Y |
| 38 | T F F Y G G C G G N K N N F V R E K Y |
| 39 | T F F Y G G C M G N K N N F V R E K Y |
| 40 | T F F Y G G S M G N K N N F V R E K Y |
| 41 | P F F Y G G C L G N R N N Y V R E K Y |
| 42 | T F F Y G G C L G N R N N F V R E K Y |
| 43 | T F F Y G G C L G N K N N Y V R E K Y |
| 44 | T F F Y G G C G G N G N N F L T A K Y |
| 45 | T F F Y G G C R G N R N N F L T A E Y |
| 46 | T F F Y G G C R G N G N N F K S A E Y |
| 47 | P F F Y G G C L G N K N N F K T A E Y |
| 48 | T F F Y G G C R G N R N N F K T E E Y |
| 49 | T F F Y G G C R G K R N N F K T E E D |
| 50 | P F F Y G G C G G N G N N F V R E K Y |
| 51 | S F F Y G G C M G N G N N F V R E K Y |
| 52 | P F F Y G G C G G N G N N F L R E K Y |
| 53 | T F F Y G G C L G N G N N F V R E K Y |
| 54 | S F F Y G G C L G N G N N Y L R E K Y |
| 55 | T F F Y G G S L G N G N N F V R E K Y |
| 56 | T F F Y G G C R G N G N N F V T A E Y |
| 57 | T F F Y G G C L G K G N N F V S A E Y |
| 58 | T F F Y G G C L G N R N N F D R A E Y |
| 59 | T F F Y G G C L G N R N N F L R E E Y |
| 60 | T F F Y G G C L G N K N N Y L R E E Y |
| 61 | P F F Y G G C G G N R N N Y L R E E Y |
| 62 | P F F Y G G S G G N R N N Y L R E E Y |
| 63 | M R P D F C L E P P Y T G P C V A R I |
| 64 | A R I I R Y F Y N A K A G L C Q T F V Y G |
| 65 | Y G G C R A K R N N Y K S A E D C M R T C G |
| 66 | P D F C L E P P Y T G P C V A R I I R Y F Y |
| 67 | T F F Y G G C R G K R N N F K T E E Y |
| 68 | K F F Y G G C R G K R N N F K T E E Y |
| 69 | T F Y Y G G C R G K R N N Y K T E E Y |
| 70 | T F F Y G G S R G K R N N F K T E E Y |
| 71 | C T F F Y G C C R G K R N N F K T E E Y |
| 72 | T F F Y G G C R G K R N N F K T E E Y C |
| 73 | C T F F Y G S C R G K R N N F K T E E Y |
| 74 | T F F Y G G S R G K R N N F K T E E Y C |
| 75 | P F F Y G G C R G K R N N F K T E E Y |
| 76 | T F F Y G G C R G K R N N F K T K E Y |
| 77 | T F F Y G G K R G K R N N F K T E E Y |
| 78 | T F F Y G G C R G K R N N F K T K R Y |
| 79 | T F F Y G G K R G K R N N F K T A E Y |
| 80 | T F F Y G G K R G K R N N F K T A G Y |
| 81 | T F F Y G G K R G K R N N F K R E K Y |
| 82 | T F F Y G G K R G K R N N F K R A K Y |
| 83 | T F F Y G G C L G N R N N F K T E E Y |
| 84 | T F F Y G C G R G K R N N F K T E E Y |
| 85 | T F F Y G G R C G K R N N F K T E E Y |
| 86 | T F F Y G G C L G N G N N F D T E E E |
| 87 | T F Q Y G G C R G K R N N F K T E E Y |
| 88 | Y N K E F G T F N T K G C E R G Y R F |
| 89 | R F K Y G G C L G N M N N F E T L E E |
| 90 | R F K Y G G C L G N K N N F L R L K Y |
| 91 | R F K Y G G C L G N K N N Y L R L K Y |
| 92 | K T K R K R K K Q R V K I A Y E E I F K N Y |
| 93 | K T K R K R K K Q R V K I A Y |
| 94 | R G G R L S Y S R R F S T S T G R |
| 95 | R R L S Y S R R R F |
| 96 | R Q I K I W F Q N R R M K W K K |
| 97 | T F F Y G G S R G K R N N F K T E E Y |
| 98 | M R P D F C L E P P Y T G P C V A R I |
| | I R Y F Y N A K A G L C Q T F V Y G G |
| | C R A K R N N F K S A E D C M R T C G G A |
| 99 | T F F Y G G C R G K R N N F K T K E Y |
| 100 | R F K Y G G C L G N K N N Y L R L K Y |
| 101 | T F F Y G G C R A K R N N F K R A K Y |
| 102 | N A K A G L C Q T F V Y G G C L A K R N N F |
| | E S A E D C M R T C G G A |
| 103 | Y G G C R A K R N N F K S A E D C M R T C G |
| | G A |
| 104 | G L C Q T F V Y G G C R A K R N N F K S A E |
| 105 | L C Q T F V Y G G C E A K R N N F K S A |
| 107 | T F F Y G G S R G K R N N F K T E E Y |
| 108 | R F F Y G G S R G K R N N F K T E E Y |
| 109 | R F F Y G G S R G K R N N F K T E E Y |
| 110 | R F F Y G G S R G K R N N F R T E E Y |
| 111 | T F F Y G G S R G K R N N F R T E E Y |
| 112 | T F F Y G G S R G R R N N F R T E E Y |
| 113 | C T F F Y G G S R G K R N N F K T E E Y |
| 114 | T F F Y G G S R G K R N N F K T E E Y C |
| 115 | C T F F Y G G S R G R R N N F R T E E Y |
| 116 | T F F Y G G S R G R R N N F R T E E Y C |

Note:
Polypeptides nos. 5, 67, 76, and 91, include the sequences of SEQ ID NOS: 5, 67, 76, and 91, respectively, and are amidated at the C-terminus.
Polypeptides nos. 107, 109, and 110 include the sequences of SEQ ID NOS: 97, 109, and 110, respectively, and are acetylated at the N-terminus.

### Modified Polypeptides

The invention also includes a polypeptide having a modification of an amino acid sequence described herein (e.g., polypeptide having a sequence described in any one of SEQ ID NOs:1-105 and 107-112 such as AngioPep-1 (SEQ ID NO:67) or AngioPep-2 (SEQ ID NO:97)). In certain embodiments, the modification does not destroy significantly a desired biological activity. In some embodiments, the modification may cause a reduction in biological activity (e.g., by at least 5%, 10%, 20%, 25%, 35%, 50%, 60%, 70%, 75%, 80%, 90%, or 95%). In other embodiments, the modification has no effect on the biological activity or may increase (e.g., by at least 5%, 10%, 25%, 50%, 100%, 200%, 500%, or 1000%) the biological activity of the original polypeptide. The modified peptide may have or may optimize one or more of the characteristics of a polypeptide of the invention, which in some instances, might be needed or desirable. Such characteristics include in vivo stability, bioavailability, toxicity, immunological activity, and immunological identity.

Polypeptides of the invention may include amino acids or sequences modified either by natural processes, such as posttranslational processing, or by chemical modification techniques known in the art. Modifications may occur anywhere in a polypeptide including the polypeptide backbone, the amino acid side-chains and the amino- or carboxy-terminus. The same type of modification may be present in the same or varying degrees at several sites in a given polypeptide, and a polypeptide may contain more than one type of modification. Polypeptides may be branched as a result of ubiquitination, and they may be cyclic, with or without branching. Cyclic, branched, and branched cyclic polypeptides may result from posttranslational natural processes or may be made synthetically. Other modifications include PEGylation, acetylation, acylation, addition of acetomidomethyl (Acm) group, ADP-ribosylation, alkylation, amidation, biotinylation, carbamoylation, carboxyethylation, esterification, covalent attachment to fiavin, covalent attachment to a heme moiety, covalent attachment of a nucleotide or nucleotide derivative, covalent attachment of drug, covalent attachment of a marker (e.g., fluorescent or radioactive), covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination.

A modified polypeptide may further include an amino acid insertion, deletion, or substitution, either conservative or non-conservative (e.g., D-amino acids, desamino acids) in the polypeptide sequence (e.g., where such changes do not substantially alter the biological activity of the polypeptide).

Substitutions may be conservative (i.e., wherein a residue is replaced by another of the same general type or group) or non-conservative (i.e., wherein a residue is replaced by an amino acid of another type). In addition, a non-naturally-occurring amino acid may substituted for a naturally-occurring amino acid (i.e., non-naturally occurring conservative amino acid substitution or a non-naturally occurring non-conservative amino acid substitution).

Polypeptides made synthetically may include substitutions of amino acids not naturally encoded by DNA (e.g., non-naturally occurring or unnatural amino acid). Examples of non-naturally occurring amino acids include D-amino acids, an amino acid having an acetylaminomethyl group attached to a sulfur atom of a cysteine, a PEGylated amino acid, the omega amino acids of the formula NH₂(CH₂)ₙCOOH wherein n is 2,6, neutral nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties.

Analogs may be generated by substitutional mutagenesis and retain the biological activity of the original polypeptide. Examples of substitutions identified as “conservative substitutions” are shown in Table 2. If such substitutions result in a change not desired, then other type of substitutions, denominated “exemplary substitutions” in Table 2, or as further described herein in reference to amino acid classes, are introduced and the products screened.

Substantial modifications in function or immunological identity are accomplished by selecting substitutions that differ significantly in their effect on maintaining (a) the structure of the polypeptide backbone in the area of the substitution, for example, as a sheet or helical conformation. (b) the charge or hydrophobicity of the molecule at the target site, or (c) the bulk of the side chain. Naturally-occurring residues are divided into groups based on common side chain properties:

(1) hydrophobic: norleucine, methionine (Met), Alanine (Ala), Valine (Val), Leucine (Leu), Isoleucine (Ile), Histidine (His), Tryptophan (Trp), Tyrosine (Tyr), Phenylalanine (Phe),

(2) neutral hydrophilic: Cysteine (Cys), Serine (Ser), Threonine (Thr)

(3) acidic/negatively charged: Aspartic acid (Asp), Glutamic acid (Glu)

(4) basic: Asparagine (Asn), Glutamine (Gln), Histidine (His), Lysine (Lys), Arginine (Arg)

(5) residues that influence chain orientation: Glycine (Gly), Proline (Pro);

(6) aromatic: Tryptophan (Trp), Tyrosine (Tyr), Phenylalanine (Phe), Histidine (His).

(7) polar: Ser, Thr, Asn, Gln

(8) basic positively charged: Arg, Lys, H, and;

(9) charged: Asp, Glu, Arg, Lys, His

Other conservative amino acid substitutions are listed in Table 2.

TABLE 2
Amino acid substitutions

| Original residue | Exemplary substitution | Conservative substitution |
|---|---|---|
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, | Leu |
| | norleucine | |
| Leu (L) | Norleucine, Ile, Val, Met, | Ile |
| | Ala, Phe | |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, | Leu |
| | norleucine | |

### Additional Analogs

The polypeptides, conjugates, and compositions of the invention may include polypeptide analogs of aprotinin known in the art. For example, U.S. Pat. No. 5,807,980 describes Bovine Pancreatic Trypsin Inhibitor (aprotinin)-derived inhibitors as well as a method for their preparation and therapeutic use, including the polypeptide of SEQ ID NO:102. These peptides have been used for the treatment of a condition characterized by an abnormal appearance or amount of tissue factor and/or factor VIIIa such as abnormal thrombosis. U.S. Pat. No. 5,780,265 describes serine protease inhibitors capable of inhibiting plasma kallikrein, including SEQ ID NO:103. U.S. Pat. No. 5,118,668 describes Bovine Pancreatic Trypsin Inhibitor variants, including SEQ ID NO:105. The aprotinin amino acid sequence (SEQ ID NO:98), the Angiopep-1 amino acid sequence (SEQ ID NO:67), and SEQ ID NO:104, as well as some sequences of biologically-active analogs may be found in International Application Publication No. WO 2004/060403. An exemplary nucleotide sequence encoding an aprotinin analog is illustrated by SEQ ID NO:106 (atgagaccag atttctgcct cgagccgccg tacactgggc cctgcaaagc tcgtatcatc cgttacttct acaatgcaaa ggcaggcctg tgtcagacct tcgtatacgg cggctgcaga gctaagcgta acaacttcaa atccgcggaa gactgcatgc gtacttgcgg tggtgcttag; Genbank accession No. X04666).

Other examples of aprotinin analogs may be found by performing a protein BLAST (Genbank: www.ncbi.nlm.nih.gov/BLAST/) using the synthetic aprotinin sequence (or portion thereof) disclosed in International Application No. PCT/CA2004/000011. Exemplary aprotinin analogs are found under accession Nos. CAA37967 (GI:58005) and 1405218C (GI:3604747).

### Preparation of Polypeptide Derivatives and Peptidomimetics

In addition to polypeptides consisting only of naturally occurring amino acids, peptidomimetics or polypeptide analogs are also encompassed by the present invention. Polypeptide analogs are commonly used in the pharmaceutical industry as non-peptide drugs with properties analogous to those of the template polypeptide. The non-peptide compounds are termed “peptide mimetics” or peptidomimetics (Fauchere et al., Infect. Immun. 54:283-287, 1986; Evans et al., J. Med. Chem. 30:1229-1239, 1987). Peptide mimetics that are structurally related to therapeutically useful peptides or polypeptides may be used to produce an equivalent or enhanced therapeutic or prophylactic effect. Generally, peptidomimetics are structurally similar to the paradigm polypeptide (i.e., a polypeptide that has a biological or pharmacological activity) such as naturally-occurring receptor-binding polypeptides, but have one or more peptide linkages optionally replaced by linkages such as —CH₂NH—, —CH₂S—, —CH₂—CH₂—, —CH═CH— (cis and trans), —CH₂SO—, —CH(OH)CH₂—, —COCH₂— etc., by methods well known in the art (Spatola, Peptide Backbone Modifications, Vega Data, 1(3):267, 1983; Spatola et al., Life Sci. 38:1243-1249, 1986; Hudson et al., Int. J. Pept. Res. 14:177-185, 1979; and Weinstein. B., 1983, Chemistry and Biochemistry, of Amino Acids, Peptides and Proteins, Weinstein eds, Marcel Dekker, New-York). Such polypeptide mimetics may have significant advantages over naturally-occurring polypeptides including more economical production, greater chemical stability, enhanced pharmacological properties (e.g., half-life, absorption, potency, efficiency), reduced antigenicity and others.

While the polypeptides described herein may efficiently target particular cell types (e.g., those described herein), their effectiveness may be reduced by the presence of proteases. Serum proteases have specific substrate requirements. The substrate must have both L-amino acids and peptide bonds for cleavage. Furthermore, exopeptidases, which represent the most prominent component of the protease activity in serum, usually act on the first peptide bond of the polypeptide and require a free N-terminus (Powell et al., Pharm. Res. 10:1268-1273, 1993). In light of this, it is often advantageous to use modified versions of polypeptides. The modified polypeptides retain the structural characteristics of the original L-amino acid polypeptides that confer biological activity with regard to IGF-1, but are advantageously not readily susceptible to cleavage by protease and/or exopeptidases.

Systematic substitution of one or more amino acids of a consensus sequence with D-amino acid of the same type (e.g., an enantiomer; D-lysine in place of L-lysine) may be used to generate more stable polypeptides. Thus, a polypeptide derivative or peptidomimetic as described herein may be all L-, all D- or mixed D, L polypeptides. The presence of an N-terminal or C-terminal D-amino acid increases the in vivo stability of a polypeptide since peptidases cannot utilize a D-amino acid as a substrate (Powell et al., Pharm. Res. 10:1268-1273, 1993). Reverse-D polypeptides are polypeptides containing

D-amino acids, arranged in a reverse sequence relative to a polypeptide containing L-amino acids. Thus, the C-terminal residue of an L-amino acid polypeptide becomes N-terminal for the D-amino acid polypeptide, and so forth. Reverse D-polypeptides retain the same tertiary conformation and therefore the same activity, as the L-amino acid polypeptides, but are more stable to enzymatic degradation in vitro and in vivo, and thus have greater therapeutic efficacy than the original polypeptide (Brady and Dodson, Nature 368:692-693, 1994; Jameson et al., Nature 368:744-746, 1994). In addition to reverse-D-polypeptides, constrained polypeptides comprising a consensus sequence or a substantially identical consensus sequence variation may be generated by methods well known in the art (Rizo and Gierasch, Ann. Rev. Biochem. 61:387-418, 1992). For example, constrained polypeptides may be generated by adding cysteine residues capable of forming disulfide bridges and, thereby, resulting in a cyclic polypeptide. Cyclic polypeptides have no free N- or C-termini. Accordingly, they are not susceptible to proteolysis by exopeptidases, although they are, of course, susceptible to endopeptidases, which do not cleave at polypeptide termini. The amino acid sequences of the polypeptides with N-terminal or C-terminal D-amino acids and of the cyclic polypeptides are usually identical to the sequences of the polypeptides to which they correspond, except for the presence of N-terminal or C-terminal D-amino acid residue, or their circular structure, respectively.

A cyclic derivative containing an intramolecular disulfide bond may be prepared by conventional solid phase synthesis while incorporating suitable S-protected cysteine or homocysteine residues at the positions selected for cyclization such as the amino and carboxy termini (Sah et al., J. Pharm. Pharmacol. 48:197, 1996). Following completion of the chain assembly, cyclization can be performed either (1) by selective removal of the S-protecting group with a consequent on-support oxidation of the corresponding two free SH-functions, to form a S-S bonds, followed by conventional removal of the product from the support and appropriate purification procedure or (2) by removal of the polypeptide from the support along with complete side chain de-protection, followed by oxidation of the free SH-functions in highly dilute aqueous solution.

The cyclic derivative containing an intramolecular amide bond may be prepared by conventional solid phase synthesis while incorporating suitable amino and carboxyl side chain protected amino acid derivatives, at the position selected for cyclization. The cyclic derivatives containing intramolecular -S-alkyl bonds can be prepared by conventional solid phase chemistry while incorporating an amino acid residue with a suitable amino-protected side chain, and a suitable S-protected cysteine or homocysteine residue at the position selected for cyclization.

Another effective approach to confer resistance to peptidases acting on the N-terminal or C-terminal residues of a polypeptide is to add chemical groups at the polypeptide termini, such that the modified polypeptide is no longer a substrate for the peptidase. One such chemical modification is glycosylation of the polypeptides at either or both termini. Certain chemical modifications, in particular N-terminal glycosylation, have been shown to increase the stability of polypeptides in human serum (Powell et al., Pharm. Res. 10:1268-1273, 1993). Other chemical modifications which enhance serum stability include, but are not limited to, the addition of an N-terminal alkyl group, consisting of a lower alkyl of from one to twenty carbons, such as an acetyl group, and/or the addition of a C-terminal amide or substituted amide group. In particular, the present invention includes modified polypeptides consisting of polypeptides bearing an N-terminal acetyl group and/or a C-terminal amide group.

Also included by the present invention are other types of polypeptide derivatives containing additional chemical moieties not normally part of the polypeptide, provided that the derivative retains the desired functional activity of the polypeptide. Examples of such derivatives include (1) N-acyl derivatives of the amino terminal or of another free amino group, wherein the acyl group may be an alkanoyl group (e.g., acetyl, hexanoyl, octanoyl) an aroyl group (e.g., benzoyl) or a blocking group such as F-moc (fluorenylmethyl-O—CO—); (2) esters of the carboxy terminal or of another free carboxy or hydroxyl group; (3) amide of the carboxy-terminal or of another free carboxyl group produced by reaction with ammonia or with a suitable amine; (4) phosphorylated derivatives; (5) derivatives conjugated to an antibody or other biological ligand and other types of derivatives.

Longer polypeptide sequences which result from the addition of additional amino acid residues to the polypeptides described herein are also encompassed in the present invention. Such longer polypeptide sequences can be expected to have the same biological activity and specificity (e.g., cell tropism and) as the polypeptides described above. While polypeptides having a substantial number of additional amino acids are not excluded, it is recognized that some large polypeptides may assume a configuration that masks the effective sequence, thereby preventing binding to a target (e.g., a member of the LRP receptor family such as LRP or LRP2). These derivatives could act as competitive antagonists. Thus, while the present invention encompasses polypeptides or derivatives of the polypeptides described herein having an extension, desirably the extension does not destroy the cell targeting activity of the polypeptides or its derivatives.

Other derivatives included in the present invention are dual polypeptides consisting of two of the same, or two different polypeptides, as described herein, covalently linked to one another either directly or through a spacer, such as by a short stretch of alanine residues or by a putative site for proteolysis (e.g., by cathepsin, see e.g., U.S. Pat. No. 5,126,249 and European Patent No. 495 049). Multimers of the polypeptides described herein consist of a polymer of molecules formed from the same or different polypeptides or derivatives thereof.

The present invention also encompasses polypeptide derivatives that are chimeric or fusion proteins containing a polypeptide described herein, or fragment thereof, linked at its amino- or carboxy-terminal end, or both, to an amino acid sequence of a different protein. Such a chimeric or fusion protein may be produced by recombinant expression of a nucleic acid encoding the protein. For example, a chimeric or fusion protein may contain at least 6 amino acids shared with one of the described polypeptides which desirably results in a chimeric or fusion protein that has an equivalent or greater functional activity.

The polypeptide derivatives described herein can be made by altering the amino acid sequences by substitution, addition, or deletion or an amino acid residue to provide a functionally equivalent molecule, or functionally enhanced or diminished molecule, as desired. The polypeptide derivatives include, but are not limited to, those containing, as primary amino acid sequence, all or part of the amino acid sequence of the polypeptides described herein (e.g., a VEGFR polypeptide 2.1, 2.2, or 2.3, or an APG-201, APG-202, APG-203, APG-204, APG-205, or APG-206 peptide, or an API-101, API-103, or API-106 peptide, or an API-401, API-402, API-403, API-404, or API-405 polypeptide) including altered sequences containing substitutions of functionally equivalent amino acid residues. For example, one or more amino acid residues within the sequence can be substituted by another amino acid of a similar polarity, which acts as a functional equivalent, resulting in a silent alteration. Substitution for an amino acid within the sequence may be selected from other members of the class to which the amino acid belongs. For example, the positively-charged (basic) amino acids include, arginine, lysine and histidine. The nonpolar (hydrophobic) amino acids include, leucine, isoleucine, alanine, phenylalanine, valine, proline, tryptophane and methionine. The uncharged polar amino acids include serine, threonine, cysteine, tyrosine, asparagine and glutamine. The negatively charged (acid) amino acids include glutamic acid and aspartic acid. The amino acid glycine may be included in either the nonpolar amino acid family or the uncharged (neutral) polar amino acid family. Substitutions made within a family of amino acids are generally understood to be conservative substitutions.

### Assays to Identify Peptidomimetics

As described above, non-peptidyl compounds generated to replicate the backbone geometry and pharmacophore display (peptidomimetics) of the polypeptides described herein often possess attributes of greater metabolic stability, higher potency, longer duration of action and better bioavailability.

The peptidomimetics compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des. 12:145, 1997). Examples of methods for the synthesis of molecular libraries can be found in the art, for example, in: DeWitt et al., (Proc. Natl. Acad. Sci. USA 90:6909, 1993); Erb et al., (Proc. Natl. Acad. Sci. USA 91:11422, 1994); Zuckermann et al., (J. Med. Chem. 37:2678, 1994); Cho et al., (Science 261:1303, 1993); Carell et al., (Angew. Chem., Int. Ed. Engl. 33:2059, 1994 and ibid 2061); and in Gallop et al., (Med. Chem. 37:1233, 1994). Libraries of compounds may be presented in solution (e.g., Houghten, Biotechniques 13:412-421, 1992) or on beads (Lam, Nature 354:82-84, 1991), chips (Fodor, Nature 364:555-556, 1993), bacteria or spores (U.S. Pat. No. 5,223,409), plasmids (Cull et al., Proc. Natl. Acad. Sci. USA 89:1865-1869, 1992) or on phage (Scott and Smith, Science 249:386-390, 1990), or luciferase, and the enzymatic label detected by determination of conversion of an appropriate substrate to product.

Once a polypeptide as described herein is identified, it may be isolated and purified by any number of standard methods including, but not limited to, differential solubility (e.g., precipitation), centrifugation, chromatography (e.g., affinity, ion exchange, size exclusion, and the like) or by any other standard techniques used for the purification of peptides, peptidomimetics, or proteins. The functional properties of an identified polypeptide of interest may be evaluated using any functional assay known in the art. Desirably, assays for evaluating downstream receptor function in intracellular signaling are used (e.g., cell proliferation).

For example, the peptidomimetics compounds of the present invention may be obtained using the following three-phase process: (1) scanning the polypeptides described herein to identify regions of secondary structure necessary for targeting the particular cell types described herein; (2) using conformationally constrained dipeptide surrogates to refine the backbone geometry and provide organic platforms corresponding to these surrogates; and (3) using the best organic platforms to display organic pharmocophores in libraries of candidates designed to mimic the desired activity of the native polypeptide. In more detail the three phases are as follows. In phase 1, the lead candidate polypeptides are scanned and their structure abridged to identify the requirements for their activity. A series of polypeptide analogs of the original are synthesized. In phase 2, the best polypeptide analogs are investigated using the conformationally constrained dipeptide surrogates. Indolizidin-2-one, indolizidin-9-one and quinolizidinone amino acids (I²aa, I⁹aa and Qaa respectively) are used as platforms for studying backbone geometry of the best peptide candidates. These and related platforms (reviewed in Halab et al., Biopolymers 55:101-122, 2000; and Hanessian et al., Tetrahedron 53:12789-12854, 1997) may be introduced at specific regions of the polypeptide to orient the pharmacophores in different directions. Biological evaluation of these analogs identifies improved lead polypeptides that mimic the geometric requirements for activity. In phase 3, the platforms from the most active lead polypeptides are used to display organic surrogates of the pharmacophores responsible for activity of the native peptide. The pharmacophores and scaffolds are combined in a parallel synthesis format. Derivation of polypeptides and the above phases can be accomplished by other means using methods known in the art.

Structure function relationships determined from the polypeptides, polypeptide derivatives, peptidomimetics or other small molecules described herein may be used to refine and prepare analogous molecular structures having similar or better properties.

Accordingly, the compounds of the present invention also include molecules that share the structure, polarity, charge characteristics and side chain properties of the polypeptides described herein.

In summary, based on the disclosure herein, those skilled in the art can develop peptides and peptidomimetics screening assays which are useful for identifying compounds for targeting an agent to particular cell types (e.g., those described herein). The assays of this invention may be developed for low-throughput, high-throughput, or ultra-high throughput screening formats. Assays of the present invention include assays which are amenable to automation.

### Nucleic Acids

The polypeptides described herein may be conjugated to any nucleic acid. As such, the polypeptides can serve as vectors to target and transport the conjugated nucleic acid to a specific cell, tissue, or organ, or across the BBB. Conjugated nucleic acids can include expression vectors (e.g., a plasmid) and therapeutic nucleic acids (e.g., RNAi agents). Nucleic acids include any type known in the art, such as double and single-stranded DNA and RNA molecules of any length, conformation, charge, or shape (i.e., linear, concatemer, circular (e.g., a plasmid), nicked circular, coiled, supercoiled, or charged. Additionally, the nucleic acid can contain 5′ and 3′ terminal modifications and include blunt and overhanging nucleotides at these termini, or combinations thereof. In certain embodiments of the invention the nucleic acid is or encodes an RNA interference sequence (e.g., an siRNA, shRNA, miRNA, or dsRNA nucleotide sequence) that can silence a targeted gene product. The nucleic acid can be, for example, a DNA molecule, an RNA molecule, or a modified form thereof.

### Expression Vectors

In certain embodiments, the nucleic acid is capable of being expressed in a cell. The nucleic may encode a polypeptide (e.g., a therapeutic polypeptide) or may encode a therapeutic nucleic acid (e.g., an RNAi agent such as those described herein). Any expression system known in the art may be used and any suitable disease may be treated using a expression system (e.g., a plasmid) known in the art. In an exemplary approach (Horton et al., Proc. Natl. Acad. Sci. USA 96:1553-1558, 1999), a plasmid encoding a cytokine (interferon alpha) is provided to a subject having a cancer. Following entry into the cell, the cytokine gene is expressed by cellular transcription and translation pathways to produce a cytokine protein that, in turn inhibits, tumor proliferation. Other approaches are described, for example, in Mahvi et al., Cancer Gene Ther. 14:717-723, 2007. Here, a plasmid expressing IL-12 was injected into metastatic tumors, thereby resulting in decreased tumor size. Because the conjugates of the invention may be capable of targeting a nucleic acid to particular cell types including cancer cells, conjugating a nucleic acid to a vector may allow for systemic delivery of such nucleic acids. Diseases such as cardiovascular disorders can also be treated using similar therapies. Growth factors such as FGF-2 can be administered to a patient suffering from myocardial ischemia using a plasmid vector encoding the growth factor. Transport of plasmid DNA to tissues such as liver may also be desirable for treating or vaccinating against cancers such as hepatoma or other liver cancer. See, e.g., Chou http://www.nature.com/cgt/journal/v13/n8/abs/7700927-aff1 et al., Cancer Gene Ther. 13:746-752, 2006.

Other approaches include using a polypeptide conjugated to a DNA plasmid that encodes a shRNA nucleotide sequence (e.g., EGFR). Upon localization in a target cell, the shRNA molecule is transcribed from the plasmid and, after processing by Dicer, results in the down-regulation of a target gene product. In another embodiment, the polypeptide vectors of the invention are conjugated to viral nucleic acid or virus particles (e.g., adenovirus, retrovirus) which carry viral genomes carrying recombinant siRNA sequences. Upon transport to the target cells or through the BBB, the viral nucleic acid or particles bind and transduce target cells. The viral genome is thus expressed in the target cell, allowing for transcription of a therapeutic molecule.

RNA Interference

RNA interference (RNAi) is a mechanism that inhibits gene expression by causing the degradation of specific RNA molecules or hindering the transcription of specific genes. In nature, RNAi targets are often RNA molecules from viruses and transposons (a form of innate immune response), although it also plays a role in regulating development and genome maintenance. Key to the mechanism of RNAi are small interfering RNA strands (siRNA), which have complementary nucleotide sequences to a targeted messenger RNA (mRNA) molecule. The siRNA directs proteins within the RNAi pathway to the targeted mRNA and degrades them, breaking them down into smaller portions that can no longer be translated into protein.

The RNAi pathway is initiated by the enzyme Dicer, which cleaves long, double-stranded RNA (dsRNA) molecules into siRNA molecules, typically about 21 to about 23 nucleotides in length and containing about 19 base pair duplexes. One of the two strands of each fragment, known as the guide strand, is then incorporated into the RNA-induced silencing complex (RISC) and pairs with complementary sequences. RISC mediates cleavage of single-stranded RNA having sequence complementary to the antisense strand of the siRNA duplex. Cleavage of the target RNA takes place in the middle of the region complementary to the antisense strand of the siRNA duplex. The outcome of this recognition event is post-transcriptional gene silencing. This occurs when the guide strand specifically pairs with a mRNA molecule and induces the degradation by Argonaute, the catalytic component of the RISC complex.

The application of RNAi technology in the present invention can occur in several ways, each resulting in functional silencing of a gene of interest (e.g., epidermal growth factor receptor (EGFR)). RNAi may be accomplished with a siRNA molecule conjugated to the vector polypeptides described herein (e.g., AngioPep-2, SEQ ID NO:97). In another embodiment, the RNAi agent is constructed containing a hairpin sequence (i.e., an shRNA, such as a 21-bp hairpin) representing a sequence directed against the gene of interest. The siRNA, shRNA, dsRNA, miRNA, or other RNAi agent is introduced to the target cell and reduces target mRNA and protein expression.

Functional gene silencing by an RNAi agent does not necessarily include complete inhibition of the targeted gene product. In some cases, marginal decreases in gene product expression caused by an RNAi agent may translate to significant functional or phenotypic changes in the host cell, tissue, organ, or animal. Therefore, gene silencing is understood to be a functional equivalent and the degree of gene product degradation to achieve silencing may differ between gene targets or host cell type. Gene silencing may decrease gene product expression by 1%, 2%, 3%, 4%, 5%, 6%, 7%, 8%, 9%, or 10%. Preferentially, gene product expression is decreased by 10%, 20%, 30%, 40%, 50%, 60%, 70%, 80%, 90%, or 100% (i.e., complete inhibition).

siRNA

Small interfering RNAs (siRNA) represent an important RNAi modality in the present invention. Certain siRNA motifs are commonly used. For example, an siRNA can be a short (usually 21-nt), double-strand of RNA (dsRNA). Many siRNA molecules have, for example, 1 or 2 nucleotide overhangs on the 3′ ends, but can also be blunt-ended. Each strand has a 5′ phosphate group and a 3′ hydroxyl (—OH) group. Most siRNA molecules are 18 to 23 nucleotides in length, however a skilled practitioner may vary this sequence length to increase or decrease the overall level of gene silencing. siRNAs can also be exogenously (i.e., artificially) introduced into cells by various methods to bring about the specific knockdown of a gene of interest. Almost any gene of which the sequence is known can thus be targeted based on sequence complementarity with an appropriately tailored siRNA. siRNA refers to a nucleic acid molecule capable of inhibiting or down-regulating gene expression in a sequence-specific manner; see, for example, Zamore et al., Cell 101:25 33 (2000); Bass, Nature 411:428-429 (2001); Elbashir et al., Nature 411:494-498 (2001); and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914. Methods of preparing a siRNA molecule for use in gene silencing are described in U.S. Pat. No. 7,078,196, which is hereby incorporated by reference.

shRNA

A short hairpin RNA (shRNA) molecule may be used in place of a siRNA to achieve targeted gene silencing. shRNA are single-stranded RNA molecules in which a tight hairpin loop structure is present, allowing complementary nucleotides within the same strand to form bonds. shRNA can be preferable to siRNA for certain applications as the hairpin structure reduces the sensitivity of the RNA molecule to nuclease degradation. Once inside a target cell, shRNA are processed and effect gene silencing by the same mechanism described above for siRNA. The cellular enzyme Dicer is responsible for cleaving shRNA molecules that enter a target cell into optimal siRNA molecules for gene silencing.

dsRNA

Double-stranded RNA (dsRNA) can also be used as an RNAi agent. Any double-stranded RNA that can be cleaved by the enzyme Dicer into smaller, optimal siRNA molecules that target a specific mRNA can be conjugated to a polypeptide of the invention for use as an RNAi agent. Methods of preparing dsRNA for use as RNAi agents are described in U.S. Pat. No. 7,056,704, which is hereby incorporated by reference.

miRNA

MicroRNAs (miRNA) represent another RNAi agent of the invention. miRNA are single-stranded RNA molecules that can silence a target gene using the same or similar mechanisms as siRNA and shRNA agents. miRNA can be conjugated to the polypeptides of the invention to silence a target gene. miRNA molecules of 21 to 23 nucleotides in length are typically the most effective for gene silencing applications, however, a skilled practitioner may vary this sequence length to increase or decrease the overall level of gene silencing.

### RNAi Gene Targets

The present invention features the silencing of a target gene in a diseased tissue or organ by treatment with a polypeptide-nucleic acid conjugate. The conjugate may be able to cross the BBB or target specific cells efficiently (e.g., hepatocytes). Once inside the cell, the RNAi agent can dissociate from the vector and enter the RNAi silencing pathway discussed above. The therapeutic potential of the present invention is realized when the mRNA molecules of a specific and targeted gene known or thought to be involved in the establishment or maintenance of the disease state (e.g., a cancer) are degraded by the RNAi agent. Examples of RNAi targets for use with the present invention include growth factors (e.g., epidermal growth factor (EGF), vascular endothelial growth factor (VEGF), transforming growth factor-beta (TGF-beta)), growth factor receptors, including receptor tyrosine kinases (e.g., EGF receptor (EGFR), including Her2/neu (ErbB), VEGF receptor (VEGFR), platelet-derived growth factor receptor (PDGFR), cytokines, chemokines, kinases, including cytoplasmic tyrosine and serine/threonine kinases (e.g., focal adhesion kinase, cyclin-dependent kinase, SRC kinases, syk-ZAP70 kinases, BTK kinases, RAF kinase, MAP kinases (including ERK), and Wnt kinases), phosphatases, regulatory GTPases (e.g., Ras protein), transcription factors (e.g., MYC), hormones and hormone receptors (e.g., estrogen and estrogen receptor), anti-apoptotic molecules (e.g., survivin, Bcl-2, Bcl-xL), oncogenes (e.g., tumor suppressor regulators such as mdm2), enzymes (e.g., superoxide dismutase 1 (SOD-1), α, β (BACE), and γ secretases, alpha-L-iduronidase, iduronate sulfatase, heparan N-sulfatase, alpha-N-acetylglucosaminidase, acetyl-CoAlpha-glucosaminide acetyltransferase, N-acetylglucosamine 6-sulfatase, N-acetylgalactosamine 4-sulfatase, beta-galactosidase, sphingomyelinase, glucocerebrosidase, alpha-galactosidase-A, ceramidase, galactosylceramidase, arylsulfatase A, aspartoacylase, phytanoyl-CoA hydroxylase, peroxin-7, beta-hexosaminidase A, aspartylglucosaminidase, fucosidase, and alpha-mannosidase, sialidase), and other proteins (e.g., Huntingtin (Htt protein), amyloid precursor protein (APP), sorting nexins (including SNX6), α-synuclein, LINGO-1, Nogo-A, and Nogo receptor 1 (NgR-1)), and glial fibrillary acidic protein. Table 3 illustrates the relationship between exemplary RNAi targets and diseases and is not meant to limit the scope of the present invention.

Exemplary RNAi sequences to silence EGFR are SEQ ID NO:117 (GGAGCUGCCCAUGAGAAAU) and SEQ ID NO:118 (AUUUCUCAUGGGCAGCUCC). Similarly, VEGF can be silenced with an RNAi molecule having the sequence, for example, set forth in SEQ ID NO:119 (GGAGTACCCTGATGAGATC). Additional RNAi sequences for use in the agents of the invention may be either commercially available (e.g., Dharmacon, Ambion) or the practitioner may use one of several publicly available software tools for the construction of viable RNAi sequences (e.g., The siRNA Selection Server, maintained by MIT/Whitehead; available at: http://jura.wi.mit.edu/bioc/siRNAext/). Examples of diseases or conditions, and RNAi target that may be useful in treatment of such diseases, are shown in Table 3.

TABLE 3
Exemplary Diseases and Target Molecules

| Disease/Condition | RNAi Target Molecules |
|---|---|
| Cancer | |
| Glioblastoma | Epidermal growth factor receptor |
| | (EGFR), Vascular endothelial growth |
| | factor (VEGF) |
| Glioma | EGFR, VEGF |
| Astrocytoma | EGFR, VEGF |
| Neuroblastoma | EGFR, VEGF |
| Lung cancer | EGFR, VEGF |
| Breast cancer | EGFR, VEGF |
| Hepatocellular carcinoma | EGFR, VEGF |
| Neurodegenerative Disease | |
| Huntington's disease | Huntingtin (Htt) |
| Parkinson's disease | Alpha-synuclein |
| Alzheimer's disease | Amyloid precursor protein (APP), |
| | Presenilin-1 or -2, Apolipoprotein E |
| | (ApoE) |
| Amyotropic lateral schlerosis | Superoxide dismutase 1 (SOD-1) |
| Multiple schlerosis | Sorting nexin-6 (SNX6), LINGO-1, |
| | Nogo-A, NgR-1, APP |
| Lysosomal Storage Disease | |
| MPS-I (Hurler, Scheie diseases) | Alpha-L-iduronidase |
| MPS-II (Hunter syndrome) | Iduronate sulfatase |
| MPS-IIIA (Sanfilippo syndrome A) | Heparan N-sulfatase |
| MPS-IIIB (Sanfilippo syndrome B) | Alpha-N-acetylglucosaminidase |
| MPS-IIIC (Sanfilippo syndrome C) | Acetyl-CoAlpha-glucosaminide |
| | acetyltransferase |
| MPS-IIID (Sanfilippo syndrome D) | N-acetylglucosamine 6-sulfatase |
| MPS-VI (Maroteaux-Lamy | N-acetylgalactosamine 4-sulfatase |
| syndrome) | |
| MPS-VII (Sly syndrome) | Beta-glucuronidase |
| Niemann-Pick disease | Sphingomyelinase |
| Gaucher's disease | Glucocerebrosidase |
| Fabry disease | Alpha-galactosidase-A |
| Farber's disease | Ceramidase |
| Krabbé disease | Galactosylceramidase |
| Metachromatic leukodystrophy | Arylsulfatase A |
| Alexander disease | Glial fibrillary acidic protein |
| Canavan disease | Aspartoacylase |
| Refsum's disease | Phytanoyl-CoA hydroxylase or |
| | peroxin-7 |
| GM1 gangliosidoses | Beta-galactosidase |
| GM2 gangliosidoses (e.g., | Beta-hexosaminidase A |
| Tay-Sachs, Sandhoff diseases) | |
| Aspartylglucosaminuria | Aspartylglucosaminidase (AGA). |
| Fucosidosis | Fucosidase |
| Mannosidosis | Alpha-mannosidase |
| Mucolipodosis (sialidosis | Sialidase |

### Modified Nucleic Acids

Modified nucleic acids (i.e., nucleotide analogs), including modified RNA molecules, may be used in the conjugates of the present invention. The modified nucleic acids can improve the half-life, stability, specificity, delivery, solubility, and nuclease resistance qualities of the nucleic acids described herein. For example, siRNA agents can be partially or completed composed of nucleotide analogs that confer the beneficial qualities described above. As described in Elmén et al., (Nucleic Acids Res. 33(1):439-447 (2005)), synthetic, RNA-like nucleotide analogs (e.g., locked nucleic acids (LNA)) can be used to construct siRNA molecules that exhibit silencing activity against a target gene product.

Modified nucleic acids include molecules in which one or more of the components of the nucleic acid, namely sugars, bases, and phosphate moieties, are different from that which occurs in nature, preferably different from that which occurs in the human body. Nucleoside surrogates are molecules in which the ribophosphate backbone is replaced with a non-ribophosphate construct that allows the bases to the presented in the correct spatial relationship such that hybridization is substantially similar to what is seen with a ribophosphate backbone, e.g., non-charged mimics of the ribophosphate backbone.

Modifications can be incorporated into any double-stranded RNA (e.g., any RNAi agent (e.g., siRNA, shRNA, dsRNA, or miRNA)). RNA-like, DNA, and DNA-like molecules described herein, It may be desirable to modify one or both of the antisense and sense strands of an nucleic acid. As nucleic acids are polymers of subunits or monomers, many of the modifications described below occur at a position which is repeated within a nucleic acid, e.g., a modification of a base, or a phosphate moiety, or the non-linking O of a phosphate moiety. In some cases the modification will occur at all of the subject positions in the nucleic acid but in many, and in fact in most, cases it will not. For example, a modification may only occur at a 3′ or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. For example, a phosphorothioate modification at a non-linking O position may only occur at one or both termini, may only occur in a terminal regions, e.g., at a position on a terminal nucleotide or in the last 2, 3, 4, 5, or 10 nucleotides of a strand, or may occur in double strand and single strand regions, particularly at termini. Similarly, a modification may occur on the sense strand, antisense strand, or both. In some cases, the sense and antisense strand will have the same modifications or the same class of modifications, but in other cases the sense and antisense strand will have different modifications, e.g., in some cases it may be desirable to modify only one strand, e.g., the sense strand.

Two prime objectives for the introduction of modifications into the nucleic acids described herein is their increased protection from degradation in biological environments and the improvement of pharmacological properties, e.g., pharmacodynamic properties, which are further discussed below. Other suitable modifications to a sugar, base, or backbone of an nucleic acid are described in PCT Application No. PCT/US2004/01193; and incorporated herein by reference. A nucleic acid can include a non-naturally occurring base, such as the bases described in PCT Application No. PCT/US2004/011822; incorporated herein by reference. A nucleic acid can include a non-naturally occurring sugar, such as a non-carbohydrate cyclic carrier molecule. Exemplary features of non-naturally occurring sugars for use in the nucleic acids described herein are described in PCT Application No. PCT/US2004/11829, and incorporated here by reference.

Any of the nucleic acids described herein can include an internucleotide linkage (e.g., the chiral phosphorothioate linkage) useful for increasing nuclease resistance. In addition, or in the alternative, a nucleic acid can include a ribose mimic for increased nuclease resistance. Exemplary internucleotide linkages and ribose mimics for increased nuclease resistance are described in U.S. Patent Application Publication No. 2005/0164235; incorporated herein by reference.

Any nucleic acid described herein can include ligand-conjugated monomer subunits and monomers for oligonucleotide synthesis. Exemplary monomers are described in U.S. Patent Application Publication No. 2005/0107325; incorporated herein by reference.

Any nucleic acid can have a ZXY structure, such as is described in U.S. Patent Application Publication No. 2005/0164235.

Any nucleic acid can be complexed with an amphipathic moiety. Exemplary amphipathic moieties for use with RNAi agents are described in U.S. Patent Application Publication No. 2005/0164235.

### Conjugation of Polypeptide and Nucleic Acid

Conjugation of the polypeptide and the nucleic acid of the invention can be accomplished by any means known in the art. The nucleic acid can be conjugated directly to the polypeptide or may be conjugated through a linker.

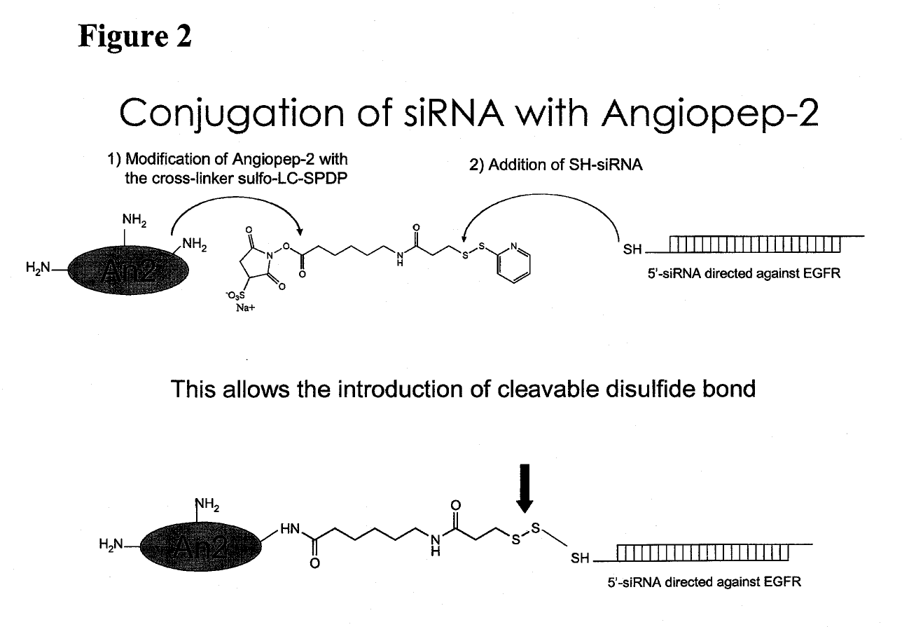

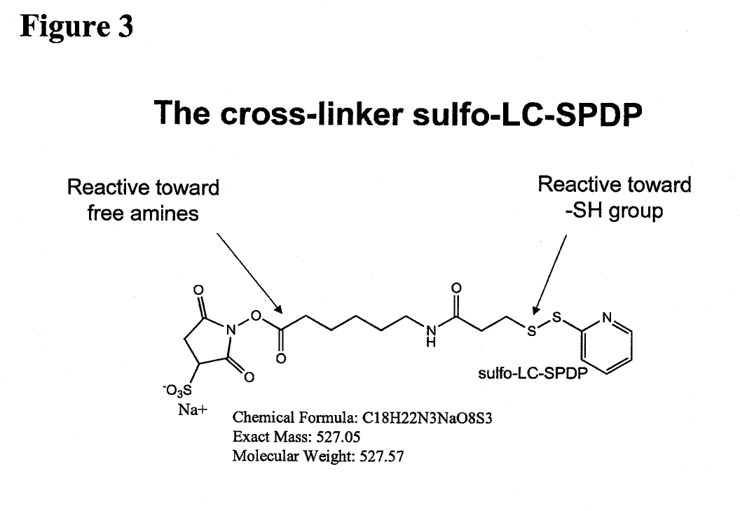

The linkage between the polypeptide and nucleic acid may be cleavable or noncleavable. In one example, a disulfide bond between the polypeptide and a siRNA molecule is introduced. This process is illustrated in FIG. 2, using AngioPep-2 (SEQ ID NO:97) and a siRNA targeting EGFR as examples. Modification of AngioPep-2 with the cross-linker sulfo-LC-SPDP allows for the conjugation of the two molecules via a cleavable disulfide bond. In general, the chemical conjugation between the polypeptide and RNAi agents of the invention is cleavable once the conjugate has entered a target cell, to allow the RNAi agent (e.g., an siRNA) to exert its gene silencing functions. Cleavable linkages include ester bonds, which can be conjugated to any free hydroxyl on the nucleic acid molecule. Other cleavable linkers include disulfide bonds. Noncleavable linkages can occur through sulfide-amino bonds.

In embodiments that use a linker, the linker may be a bifunctional linker (e.g., a homobiofunctional or heterobifunctional linker). Heterobifunctional cross-linkers include EMCS ([N-ε-maleimidocaproyloxy] succinimide ester), maleimido-hexanoic acid (MHA), MBS (m-maleimidobenzoyl-N-hydroxysuccinimide ester), Sulfo MBS (m-maleimidobenzoyl-N-hydroxysulfosuccinimide ester), GMBS (N-γ-maleimidobutyryloxysuccinimide ester), sulfo GMBS (N-γ-maleimidobutyryloxysulfosuccinimide ester), EMCH(N-(γ-maleimidocaproic acid) hydrazide), EMCS(N-(ε-maleimidocaproyloxy) succinimide ester), sulfo EMCS(N-(ε-maleimidocaproyloxy) sulfo succinimide ester, PMPI (N-(p-maleimidophenyl) isocyanate, SIAB (N-succinimidyl(4-iodoacetyl)aminobenzoate), SMCC (succinimidyl 4-(N-maleimidomethyl)cyclohexane-1-carboxylate), SMPB (succinimidyl 4-(p-maleimidophenyl) butyrate, sulfo SIAB (N-sulfosuccinimidyl(4-iodoacetyl)aminobenzoate), sulfo SMCC (sulfosuccinimidyl 4-(N-maleimidomethyl)cyclohexane-1-carboxylate), sulfo SMPB (sulfosuccinimidyl 4-(p-maleimidophenyl) butyrate), EDC (1-ethyl-3-(3-dimethylaminopropyl) carbodiimide hydrochloride), MAL-PEG-SCM (maleimide PEG succinimidyl carboxymethyl), ABH (p-azidobenzoyl hydrazide, ANB-NOS(N-5-azido-2-nitrobenzyloxysuccinimide), APDP(N-(4-[p-azidosalicylamido]butyl)-3′-(2′-pyridyldithio) propionamide), NHS-ASA (N-hydroxysuccinimidyl-4-azidosalicyclic acid), sulfo HSAB (N-hydroxysulfosuccinimidyl-4-azidobenzoate), sulfo SAED (sulfosuccinimidyl 2-(7-amino-4-methylcoumarin-3-acetamido)ethyl-1,3-dithiopropionate), sulfo SAND (sulfosuccinimidyl 2-(m-azido-o-nitrobenzamido)-ethyl-1,3′-dithiopropionate), sulfo SANPAH (sulfosuccinimidyl 6-(4′-azido-2′-nitrophenylamino) hexanoate, sulfo SADP (sulfosuccinimidyl (4-azidophenyl)-1,3′-dithiopropionate), and sulfo SASD (sulfosuccinimidyl-2-(p-azidosalicylamido)ethyl-1,3-dithiopropionate). Exemplary homobifunctional crosslinkers include BSOCOES (bis(2-[succinimidooxycarbonyloxy]ethyl) sulfone), DPDPB (1,4-di-(3′-[2′ pyridyldithio]-propionamido) butane), DSS (disuccinimidyl suberate), DST (disuccinimidyl tartrate), sulfo DST (sulfodisuccinimidyl tartrate), DSP (dithiobis(succinimidyl propionate)), DTSSP (3,3′-dithiobis(sulfosuccinimidyl propionate)), EGS (ethylene glycol bis(succinimidyl succinate)), and BASED (bis(β-[4-azidosalicylamido]-ethyl) disulfide).

In one example, a hydroxyl (e.g., on an siRNA molecule) is cleavably linked to an amine group (e.g., on a peptide vector) using an acid anhydride linker (e.g. succinic anhydride and glutaric anhydride).

In some cases, it is advantageous to conjoin the sense strand of the siRNA, shRNA, or dsRNA molecule to the polypeptide, as the antisense strand would first require phosphorylation prior to gene silencing.

Other methods and cross-linkers can be used to conjoin the polypeptides and RNAi agents of the invention. For example, a 5′ or 3′ thiol-containing siRNA sense strand can be linked by a disulfide bond to a cysteine residue placed at either the amino or carboxy terminus of the polypeptide. Muratovska et al., (FEBS Letters 558:63-68 (2004)) and Turner et al., (Blood Cells, Molecules, and Diseases 38:1-7 (2007)) provide exemplary chemical bonding methods for conjugating polypeptides to RNA molecules and are hereby incorporated by reference.

### Gene Therapy Modalities

In addition to administration of the polypeptide-nucleic acid conjugates to a subject, the present invention includes the addition of other gene therapy modalities to improve the transport to and specificity for targeted cells, tissues, or organs.

### Lipoplexes and Polyplexes

To improve the delivery of the a conjugate of the invention into the cell, the nucleic acid must be protected from damage and its entry into the cell must be facilitated. To this end, new molecules, lipoplexes and polyplexes, have been created that have the ability to protect nucleic acids from undesirable degradation during the transfection process. For example, a conjugate of the invention can be covered with lipids in an organized structure like a micelle or a liposome. When the organized structure is complexed with a nucleic acid it is called a lipoplex. There are three types of lipids, anionic (negatively-charged), neutral, or cationic (positively-charged). Lipoplexes that utilize cationic lipids have proven utility for gene transfer. Cationic lipids, due to their positive charge, naturally complex with the negatively-charged nucleic acids. Also as a result of their charge they interact with the cell membrane, endocytosis of the lipoplex occurs and the polypeptide-nucleic acid conjugate is released into the cytoplasm. The cationic lipids also protect against degradation of the nucleic acid by the cell.

Complexes of polymers with nucleic acids are called polyplexes. Most polyplexes consist of cationic polymers and their production is regulated by ionic interactions. One large difference between the methods of action of polyplexes and lipoplexes is that polyplexes cannot release their nucleic acid contents into the cytoplasm, so to this end, co-transfection with endosome-lytic agents (to lyse the endosome that is made during endocytosis) such as inactivated adenovirus must occur. However this isn't always the case, polymers such as polyethylenimine have their own method of endosome disruption as does chitosan and trimethylchitosan.

### Hybrid Methods

Some hybrid methods combine two or more techniques and can be useful for administering the conjugates of the invention to a cell, tissue, or organ of a subject. Virosomes, for example, combine liposomes with an inactivated virus. This has been shown to have more efficient gene transfer in respiratory epithelial cells than either viral or liposomal methods alone. Other methods involve mixing other viral vectors with cationic lipids or hybridising viruses.

### Dendrimers

A dendrimer is a highly branched macromolecule with a spherical shape. The surface of the particle may be functionalized in many ways and many of the properties of the resulting construct are determined by its surface. In particular it is possible to construct a cationic dendrimer (i.e. one with a positive surface charge). When in the presence of genetic material such as DNA or RNA, charge complimentarity leads to a temporary association of the nucleic acid with the cationic dendrimer. On reaching its destination the dendrimer-nucleic acid complex is then taken into the cell via endocytosis.

In recent years the benchmark for transfection agents has been cationic lipids. Limitations of these competing reagents have been reported to include: the lack of ability to transfect a number of cell types, the lack of robust active targeting capabilities, incompatibility with animal models, and toxicity. Dendrimers offer robust covalent construction and extreme control over molecule structure, and therefore size. Together these give compelling advantages compared to existing approaches.

### Cancer

The compounds, conjugates, and compositions of the invention can be used to treat any cancer, but, in the case of conjugates including a vector that is efficiently transported across the BBB, are particularly useful for the treatment of brain cancers and other cancers protected by the BBB. These include astrocytoma, pilocytic astrocytoma, dysembryoplastic neuroepithelial tumor, oligodendrogliomas, ependymoma, glioblastoma multiforme, mixed gliomas, oligoastrocytomas, medulloblastoma, retinoblastoma, neuroblastoma, germinoma and teratoma. Other types of cancer include hepatocellular carcinoma, breast cancer, cancers of the head and neck including various lymphomas such as mantle cell lymphoma, non-Hodgkins lymphoma, adenoma, squamous cell carcinoma, laryngeal carcinoma, cancers of the retina, cancers of the esophagus, multiple myeloma, ovarian cancer, uterine cancer, melanoma, colorectal cancer, bladder cancer, prostate cancer, lung cancer (including non-small cell lung carcinoma), pancreatic cancer, cervical cancer, head and neck cancer, skin cancers, nasopharyngeal carcinoma, liposarcoma, epithelial carcinoma, renal cell carcinoma, gallbladder adenocarcinoma, parotid adenocarcinoma, endometrial sarcoma, multidrug resistant cancers; and proliferative diseases and conditions, such as neovascularization associated with tumor angiogenesis, macular degeneration (e.g., wet/dry AMD), corneal neovascularization, diabetic retinopathy, neovascular glaucoma, myopic degeneration and other proliferative diseases and conditions such as restenosis and polycystic kidney disease.

### Neurodegenerative Disease

Because the polypeptides described herein are capable of transporting an agent across the BBB, the compounds, conjugates, and compositions of the invention are also useful for the treatment of neurodegenerative diseases or other conditions affecting the mammalian brain, central nervous system (CNS), the peripheral nervous system, or the autonomous nervous system wherein neurons are lost or deteriorate. Many neurodegenerative diseases are characterized by ataxia (i.e., uncoordinated muscle movements) and/or memory loss. Neurodegenerative diseases include Alexander disease, Alper disease, Alzheimer's disease, amyotrophic lateral sclerosis (ALS; i.e., Lou Gehrig's disease), ataxia telangiectasia, Batten disease (Spielmeyer-Vogt-Sjogren-Batten disease), bovine spongiform encephalopathy (BSE), Canavan disease, Cockayne syndrome, corticobasal degeneration, Creutzfeldt-Jakob disease, Huntington's disease, HIV-associated dementia, Kennedy's disease, Krabbé disease, Lewy body dementia, Machado-Joseph disease (Spinocerebellar ataxia type 3), multiple sclerosis, multiple system atrophy, narcolepsy, neuroborreliosis, Parkinson's disease, Pelizaeus-Merzbacher disease, Pick's disease, primary lateral sclerosis, prion diseases, Refsum's disease, Schilder's disease (i.e., adrenoleukodystrophy), schizophrenia, spinocerebellar ataxia, spinal muscular atrophy, Steele-Richardson, Olszewski disease, and tabes dorsalis.

### Lysosomal Storage Disorders

The compounds, conjugates, and compositions of the invention may be used to treat a lysosomal storage disease or disorder, many of which affect the central nervous system (CNS) and cause or exacerbate neurodegenerative disease. Lysosomal storage diseases include any of the mucopolysaccharidoses (MPS; including MPS-I (Hurler syndrome, Scheie syndrome), MPS-II (Hunter syndrome), MPS-IIIA (Sanfilippo syndrome A), MPS-IIIB (Sanfilippo syndrome B), MPS-IIIC (Sanfilippo syndrome C), MPS-IIID (Sanfilippo syndrome D), MPS-IV (Morquio syndrome), MPS-VI (Maroteaux-Lamy syndrome), MPS-VII (Sly syndrome), and MPS-IX (hyaluronidase deficiency)), lipidoses (including Gaucher' disease, Niemann-Pick disease, Fabry disease, Farber's disease, and Wolman's disease), gangliosidoses (including GM1 and GM2 gangliosidoses, Tay-Sachs disease, and Sandhoff disease), leukodystrophies (including adrenoleukodystrophy (i.e., Schilder's disease), Alexander disease, metachromatic leukodystrophy, Krabbé disease, Pelizaeus-Merzbacher disease, Canavan disease, childhood ataxia with central hypomyelination (CACH), Refsum's disease, and cerebrotendineous xanthomatosis), mucolipidoses (ML; including ML-I (sialidosis), ML-II (I-cell disease), ML-III (pseudo-Hurler polydystrophy), and ML-IV), and glycoproteinoses (including aspartylglucosaminuria, fucosidosis, and mannosidosis).

### Additional Indications

The polypeptide-nucleic acid conjugates of the invention can also be used to treat diseases found in other organs or tissues. For example, AngioPep-7 (SEQ ID NO:112) is efficiently transported into liver, lung, kidney, spleen, and muscle cells, allowing for the preferential treatment of diseases associated with these tissues (e.g., hepatocellular carcinoma and lung cancer). The compositions and methods of the present invention may also be used to treat genetic disorders, such as Down syndrome (i.e., trisomy 21), where down-regulation of particular gene transcripts may be useful.

### Administration and Dosage

The present invention also relates pharmaceutical compositions that contain a therapeutically effective amount of a polypeptide-nucleic acid conjugate. The composition can be formulated for use in a variety of drug delivery systems. One or more physiologically acceptable excipients or carriers can also be included in the composition for proper formulation. Suitable formulations for use in the present invention are found in Remington's Pharmaceutical Sciences, Mack Publishing Company, Philadelphia, Pa., 17th ed., 1985. For a brief review of methods for drug delivery, see, e.g., Langer, Science 249:1527-1533, 1990.

The pharmaceutical compositions are intended for parenteral, intranasal, topical, oral, or local administration, such as by a transdermal means, for prophylactic and/or therapeutic treatment. The pharmaceutical compositions can be administered parenterally (e.g., by intravenous, intramuscular, or subcutaneous injection), or by oral ingestion, or by topical application or intraarticular injection at areas affected by the vascular or cancer condition. Additional routes of administration include intravascular, intra-arterial, intratumor, intraperitoneal, intraventricular, intraepidural, as well as nasal, ophthalmic, intrascleral, intraorbital, rectal, topical, or aerosol inhalation administration. Sustained release administration is also specifically included in the invention, by such means as depot injections or erodible implants or components. Thus, the invention provides compositions for parenteral administration that comprise the above mention agents dissolved or suspended in an acceptable carrier, preferably an aqueous carrier, e.g., water, buffered water, saline, PBS, and the like. The compositions may contain pharmaceutically acceptable auxiliary substances as required to approximate physiological conditions, such as pH adjusting and buffering agents, tonicity adjusting agents, wetting agents, detergents and the like. The invention also provides compositions for oral delivery, which may contain inert ingredients such as binders or fillers for the formulation of a tablet, a capsule, and the like. Furthermore, this invention provides compositions for local administration, which may contain inert ingredients such as solvents or emulsifiers for the formulation of a cream, an ointment, and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above mentioned agent or agents, such as in a sealed package of tablets or capsules. The composition in solid form can also be packaged in a container for a flexible quantity, such as in a squeezable tube designed for a topically applicable cream or ointment.

The compositions containing an effective amount can be administered for prophylactic or therapeutic treatments. In prophylactic applications, compositions can be administered to a patient with a clinically determined predisposition or increased susceptibility to development of a tumor or cancer, or neurodegenerative disease. Compositions of the invention can be administered to the patient (e.g., a human) in an amount sufficient to delay, reduce, or preferably prevent the onset of clinical disease or tumorigenesis. In therapeutic applications, compositions are administered to a patient (e.g., a human) already suffering from a cancer or neurodegenerative disease in an amount sufficient to cure or at least partially arrest the symptoms of the condition and its complications. An amount adequate to accomplish this purpose is defined as a “therapeutically effective dose,” an amount of a compound sufficient to substantially improve some symptom associated with a disease or a medical condition. For example, in the treatment of cancer, neurodegenerative disease, or lysosomal storage disease, an agent or compound which decreases, prevents, delays, suppresses, or arrests any symptom of the disease or condition would be therapeutically effective. A therapeutically effective amount of an agent or compound is not required to cure a disease or condition but will provide a treatment for a disease or condition such that the onset of the disease or condition is delayed, hindered, or prevented, or the disease or condition symptoms are ameliorated, or the term of the disease or condition is changed or, for example, is less severe or recovery is accelerated in an individual. Amounts effective for this use may depend on the severity of the disease or condition and the weight and general state of the patient, but generally range from about 0.5 mg to about 3000 mg of the agent or agents per dose per patient. Suitable regimes for initial administration and booster administrations are typified by an initial administration followed by repeated doses at one or more hourly, daily, weekly, or monthly intervals by a subsequent administration. The total effective amount of an agent present in the compositions of the invention can be administered to a mammal as a single dose, either as a bolus or by infusion over a relatively short period of time, or can be administered using a fractionated treatment protocol, in which multiple doses are administered over a more prolonged period of time (e.g., a dose every 4-6, 8-12, 14-16, or 18-24 hours, or every 2-4 days, 1-2 weeks, once a month). Alternatively, continuous intravenous infusion sufficient to maintain therapeutically effective concentrations in the blood are contemplated.

The therapeutically effective amount of one or more agents present within the compositions of the invention and used in the methods of this invention applied to mammals (e.g., humans) can be determined by the ordinarily-skilled artisan with consideration of individual differences in age, weight, and the condition of the mammal. The agents of the invention are administered to a subject (e.g. a mammal, such as a human) in an effective amount, which is an amount that produces a desirable result in a treated subject (e.g. the slowing or remission of a cancer or neurodegenerative disorder). Such therapeutically effective amounts can be determined empirically by those of skill in the art.

The patient may also receive an agent in the range of about 0.1 to 3,000 mg per dose one or more times per week (e.g., 2, 3, 4, 5, 6, or 7 or more times per week), 0.1 to 2,500 (e.g., 2,000, 1,500, 1,000, 500, 100, 10, 1, 0.5, or 0.1) mg dose per week. A patient may also receive an agent of the composition in the range of 0.1 to 3,000 mg per dose once every two or three weeks.

Single or multiple administrations of the compositions of the invention comprising an effective amount can be carried out with dose levels and pattern being selected by the treating physician. The dose and administration schedule can be determined and adjusted based on the severity of the disease or condition in the patient, which may be monitored throughout the course of treatment according to the methods commonly practiced by clinicians or those described herein.

The carrier and conjugates of the present invention may be used in combination with either conventional methods of treatment or therapy or may be used separately from conventional methods of treatment or therapy.

When the conjugates of this invention are administered in combination therapies with other agents, they may be administered sequentially or concurrently to an individual. Alternatively, pharmaceutical compositions according to the present invention may be comprised of a combination of a carrier-agent conjugate of the present invention in association with a pharmaceutically acceptable excipient, as described herein, and another therapeutic or prophylactic agent known in the art.

### Further Conjugation

The polypeptide-nucleic acid conjugate of the present invention may be further linked to another agent, such as a therapeutic agent, a detectable label, or any other agent described herein. The conjugate may be labeled with a detectable label such as a radioimaging agent, such as those emitting radiation, for detection of a disease or condition. In other embodiments, the carrier or functional derivative thereof of the present invention or mixtures thereof may be linked to a therapeutic agent, to treat a disease or condition, or may be linked to or labeled with mixtures thereof. Treatment may be effected by administering a polypeptide-nucleic acid conjugate of the present invention that has been further conjugated to a therapeutic compound to an individual under conditions which allow transport of the agent across the blood-brain barrier or to other cells or tissues where such treatment is beneficial.

A therapeutic agent as used herein may be a drug, a medicine, an agent emitting radiation, a cellular toxin (for example, a chemotherapeutic agent) and/or biologically active fragment thereof, and/or mixtures thereof to allow cell killing or it may be an agent to treat, cure, alleviate, improve, diminish or inhibit a disease or condition in an individual treated. A therapeutic agent may be a synthetic product or a product of fungal, bacterial or other microorganism, such as mycoplasma, viral etc., animal, such as reptile, or plant origin. A therapeutic agent and/or biologically active fragment thereof may be an enzymatically active agent and/or fragment thereof, or may act by inhibiting or blocking an important and/or essential cellular pathway or by competing with an important and/or essential naturally occurring cellular component.

Examples of radioimaging agents emitting radiation (detectable radio-labels) that may be suitable are exemplified by indium-111, technitium-99, or low dose iodine-131. Detectable labels, or markers, for use in the present invention may be a radiolabel, a fluorescent label, a nuclear magnetic resonance active label, a luminescent label, a chromophore label, a positron emitting isotope for PET scanner, chemiluminescence label, or an enzymatic label. Fluorescent labels include but are not limited to, green fluorescent protein (GFP), fluorescein, and rhodamine. Chemiluminescence labels include but are not limited to, luciferase and β-galactosidase. Enzymatic labels include but are not limited to peroxidase and phosphatase. A histamine tag may also be a detectable label. For example, conjugates may comprise a carrier moiety and an antibody moiety (antibody or antibody fragment) and may further comprise a label. The label may be for example a medical isotope, such as for example and without limitation, technetium-99, iodine-123 and -131, thallium-201, gallium-67, fluorine-18, indium-111, etc.

An agent may be releasable from the polypeptide-nucleic acid conjugate after transport across the blood-brain barrier, for example, by enzymatic cleavage or breakage of a chemical bond between the vector and the agent. The released agent may then function in its intended capacity in the absence of the vector.

Covalent modifications of the polypeptide-nucleic acid conjugate are included within the scope of this invention. A chemical derivative may be conveniently prepared by direct chemical synthesis, using methods well known in the art. Such modifications may be, for example, introduced into a polypeptide, agent, or polypeptide-agent conjugate by reacting targeted amino acid residues with an organic derivatizing agent that is capable of reacting with selected side chains or terminal residues. A vector chemical derivative may be able, e.g., to cross the blood-brain barrier and be attached to or conjugated with another agent, thereby transporting the agent across the blood-brain barrier. The polypeptide-nucleic acid agent of the invention may be joined (i.e., conjugated) without limitation, through sulfhydryl groups, amino groups (amines) and/or carbohydrates to suitable detectable labels or therapeutic agents. Homobifunctional and heterobifunctional cross-linkers (conjugation agents) are available from many commercial sources. Regions available for cross-linking may be found on the carriers of the present invention. The cross-linker may comprise a flexible arm, such as for example, a short arm (<2 carbon chain), a medium-size arm (from 2-5 carbon chain), or a long arm (³ 6 carbon chain). Exemplary cross-linkers include BS3 ([Bis(sulfosuccinimidyl)suberate]; BS3 is a homobifunctional N-hydroxysuccinimide ester that targets accessible primary amines), NHS/EDC(N-hydroxysuccinimide and N-ethyl-′(dimethylaminopropyl)carbodimide; NHS/EDC allows for the conjugation of primary amine groups with carboxyl groups), sulfo-EMCS ([N-e-Maleimidocaproic acid]hydrazide; sulfo-EMCS are heterobifunctional reactive groups (maleimide and NHS-ester) that are reactive toward sulfhydryl and amino groups), hydrazide (most proteins contain exposed carbohydrates and hydrazide is a useful reagent for linking carboxyl groups to primary amines), and SATA (N-succinimidyl-S-acetylthioacetate; SATA is reactive towards amines and adds protected sulfhydryls groups).

The following examples are intended to illustrate, rather than limit, the invention.

### EXAMPLES

### Example 1

### Polypeptide-Nucleic Acid Conjugation

35 μM of single-stranded RNA oligonucleotides encoding an epidermal growth factor receptor (EGFR) siRNA sequence that contains 5′ thiol groups are incubated in annealing buffer (100 mM potassium acetate, 30 mM HEPES-KOH at pH 7.2, 2 mM magnesium acetate) for 1 min at 90° C. followed by 1 h incubation at 37° C. Annealed siRNA oligonucleotides are desalted by incubating the hybridization mix for 7 min on ice in a pre-set 1% agarose in 100 mM glucose well in an Eppendorf tube (by leaving a 100 μL tip in the molten agarose mix and allowing it to set). The desalted siRNA molecules are supplemented with 1 volume of reaction buffer (10 mM HEPES, 1 mM EDTA, pH 8.0) to adjust the final concentration of the siRNAs to 17.5 μM. Equimolar amounts of EGFR siRNA, AngioPep-2 polypeptide, and the thiol oxidant diamide (Sigma, USA) are mixed and incubated for 1 h at 40° C. The polypeptide-nucleic acid conjugate/diamide solution is mixed with culture media and applied to a target cell, tissue, organ, or patient.

### Example 2

### N-terminal and C-terminal Conjugation of siRNA to a Peptide Vector

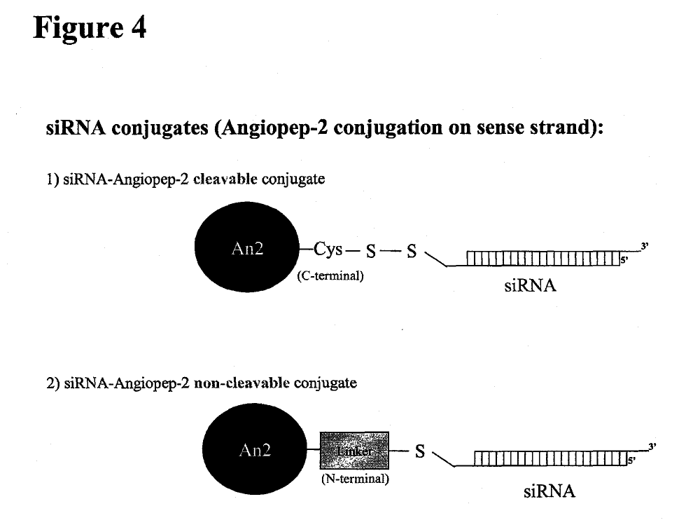

As shown in FIG. 4, a peptide vector having an N-terminal or C-terminal cysteine (e.g., SEQ ID NOS:113 and 114) can be conjugated to an SH-siRNA directly or through a linker. Depending on the linker chosen, the linkage can be cleavable or non-cleavable. Here, the peptide vector is conjugated to the sense strand of the siRNA duplex.

### Example 3

### Activity of siRNA Conjugates

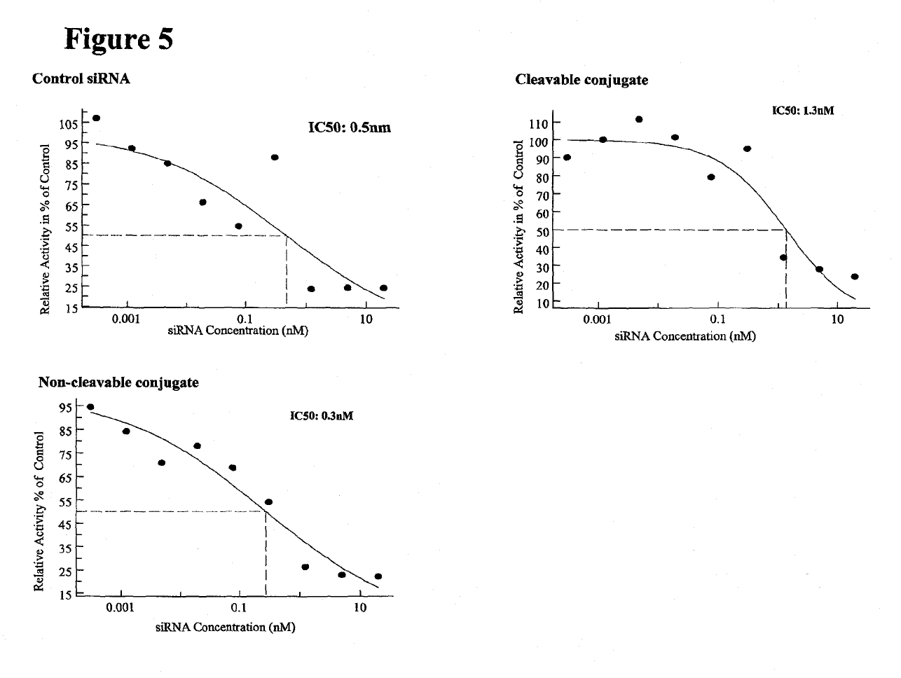

The cleavable conjugate and non-cleavable siRNA conjugates were tested for silencing activity following transfection into a test system (FIG. 5). Conjugation of Angiopep-2 does not significantly affect the silencing activity of siRNA, as both linkers have IC₅₀ values within 2-3 fold of that of the unconjugated siRNA. This silencing activity thus appears independent of the type of linker used (cleavable or non-cleavable).

### Example 4

### Transport of siRNA Conjugates Across the BBB

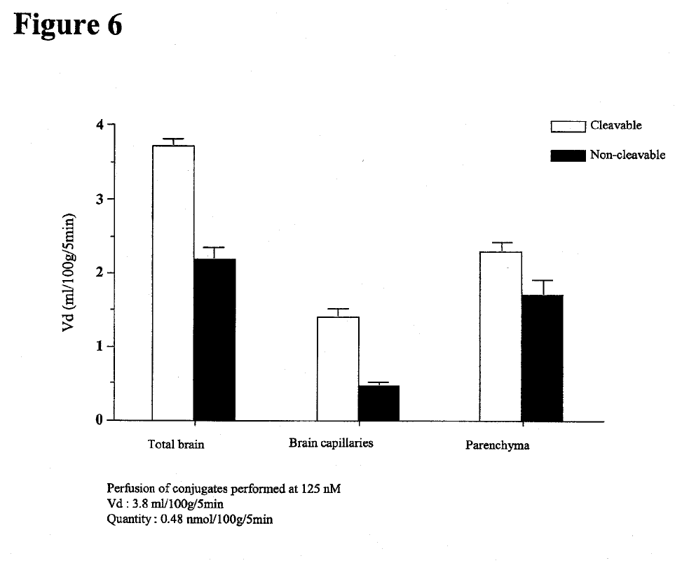

Transport of the conjugates was measured in vivo using in situ brain perfusion in mice. It was demonstrated that the siRNA-Angiopep-2 conjugates are efficiently transported across the BBB. The amount present in the brain parenchyma was determined following brain capillary depletion (FIG. 6).

### Example 5

### Additional Strategies for Conjugation of siRNA to Peptide Vectors

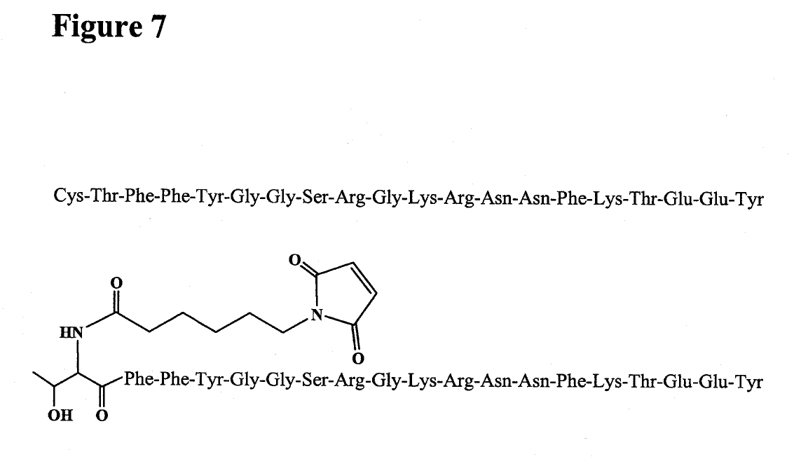

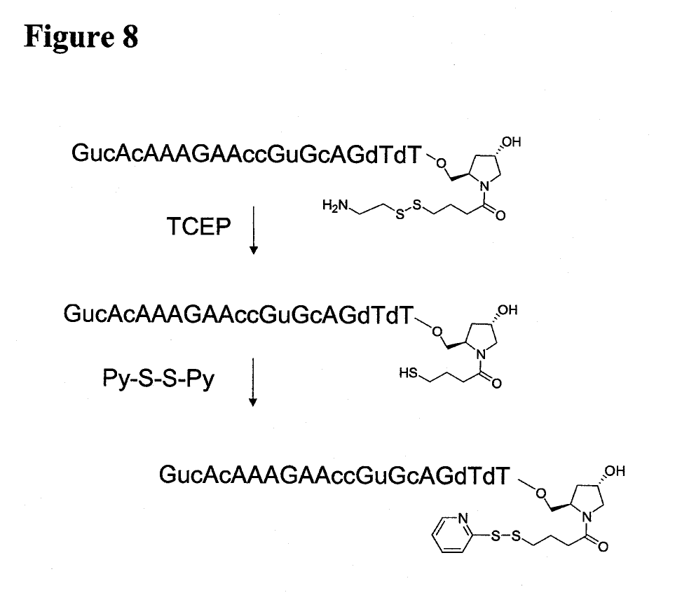

In another example, Cys-Angiopep-2 (SEQ ID NO:113) or Angiopep-2 derivatized at its N-terminal amine with 6-maleimidohexanoic acid was used as a peptide vector (FIG. 7). These peptides were conjugated to the exemplary siRNA sense strand constructs. siRNA molecules with activated disulfides were produced from siRNA derivitized as shown. Briefly, the siRNA molecule was treated with tris(2-carboxyethyl) phosphine (TCEP), to produce the free thiol, and then activated with 2,2′-dipyridyl disulfide, to form the activated compound (FIG. 8).

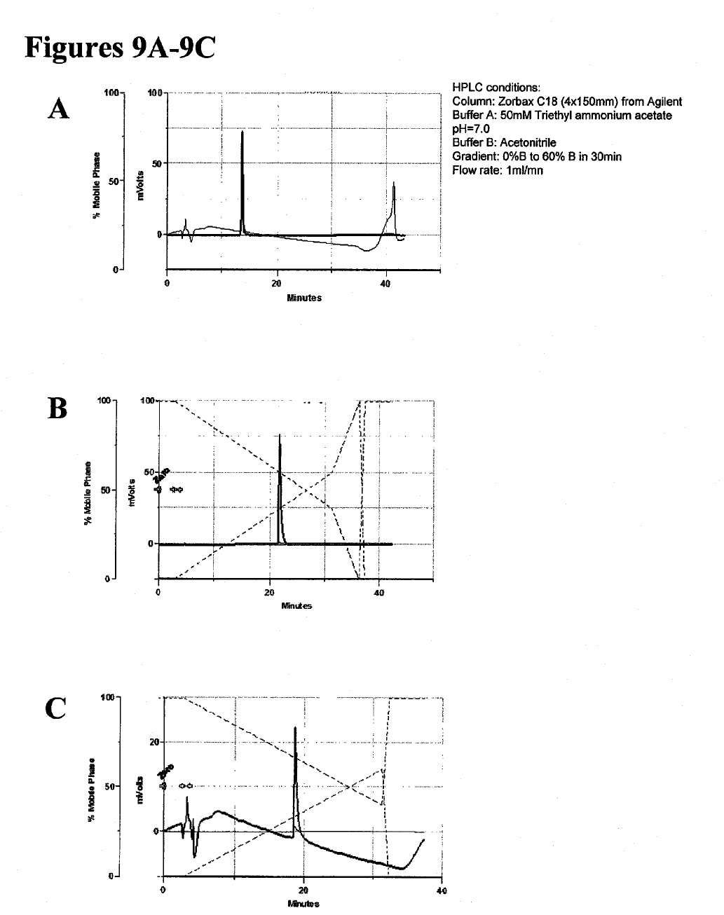

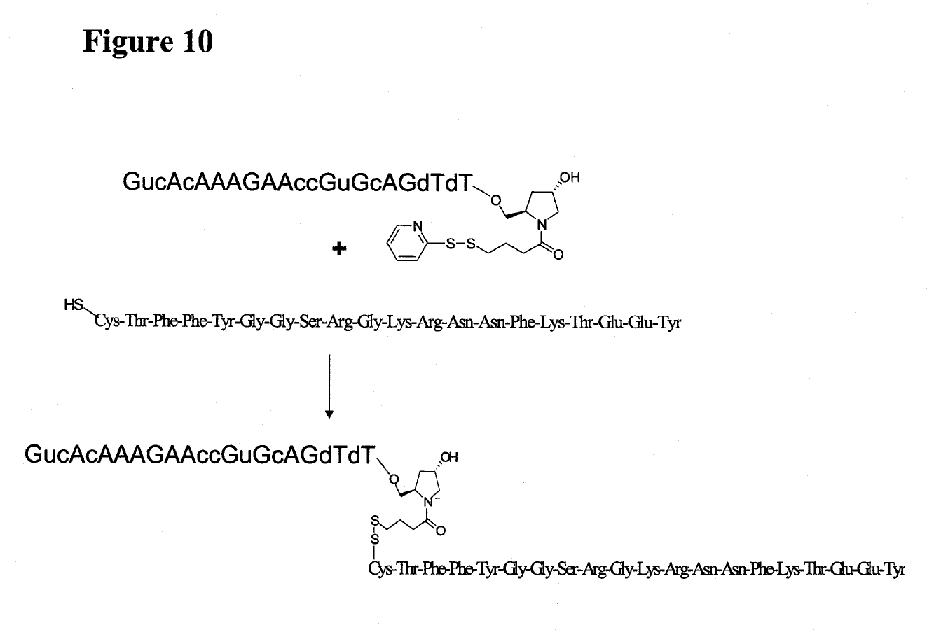

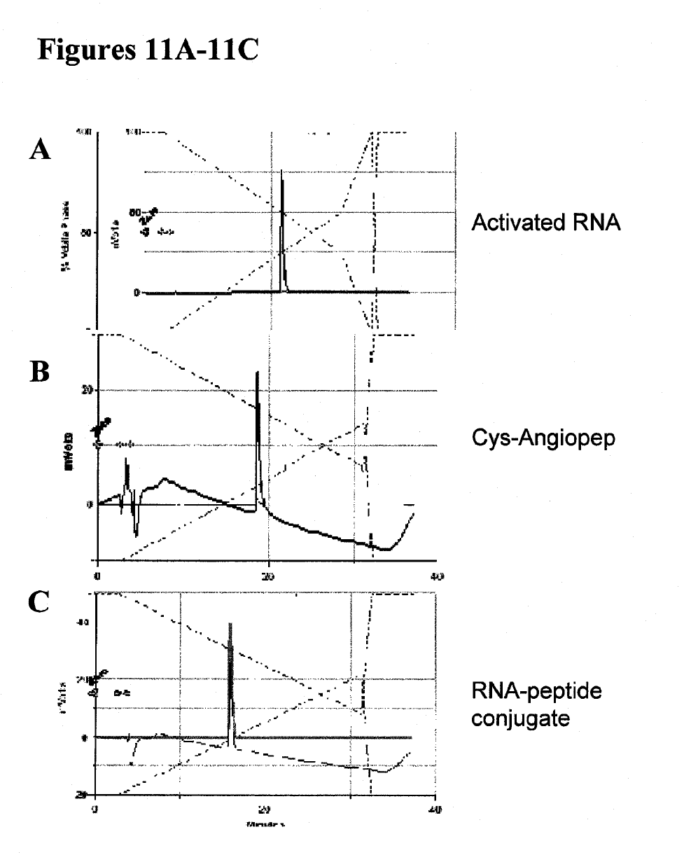

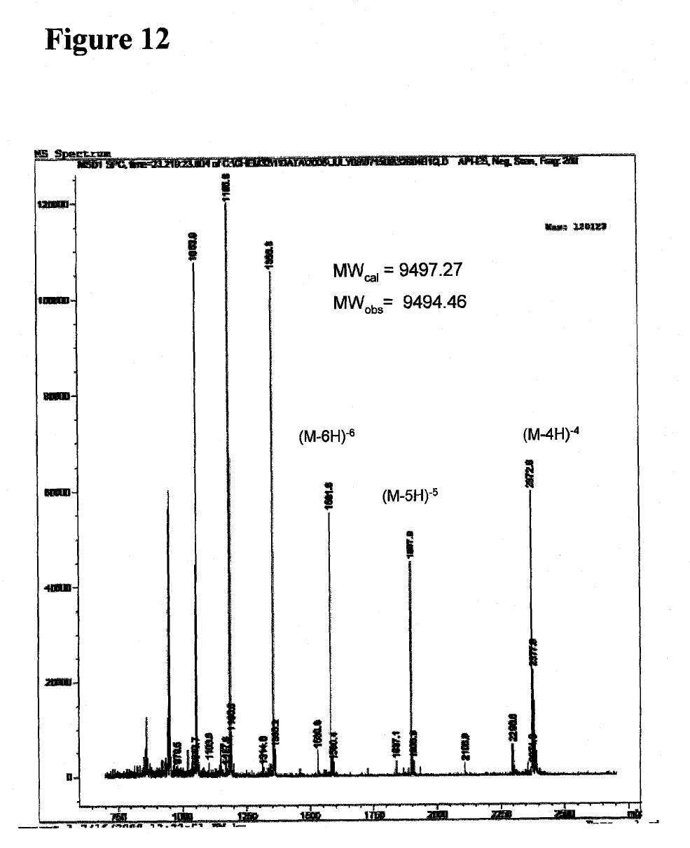

HPLC traces of the siRNA with a free thiol, synthesis of the activated siRNA, and the Cys-Angiopep-2 molecule are also shown (FIGS. 9A-9C). The activated siRNA was reacted with the Cys-Angiopep-2 to form an siRNA conjugate (FIG. 10). HPLC traces of the activated siRNA, Cys-Angiopep-2, and the resulting conjugate are shown in FIGS. 11A-11C. Mass spectroscopy was used to confirm formation of the conjugate (FIG. 12).

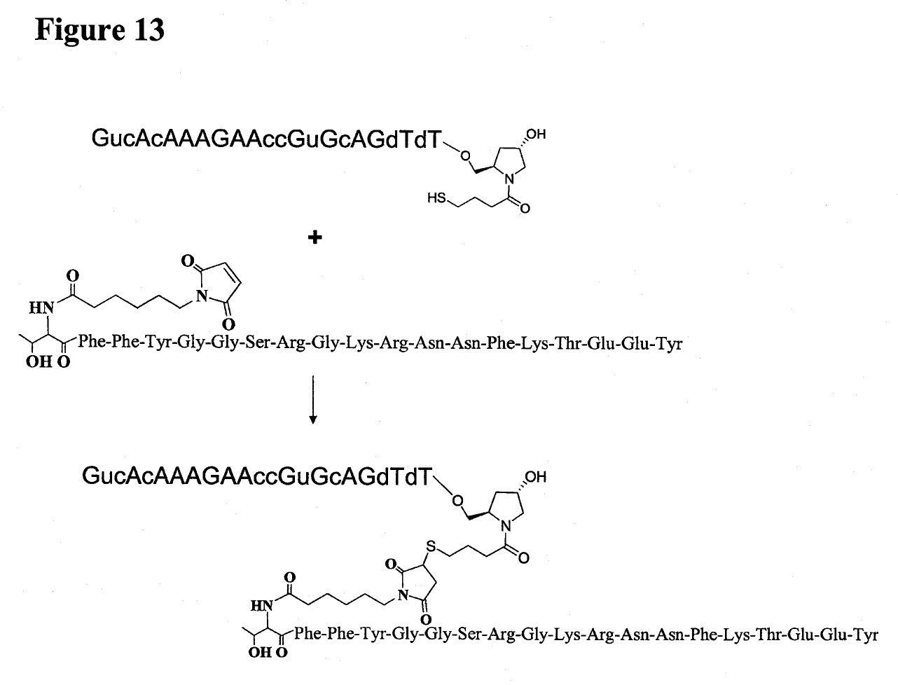

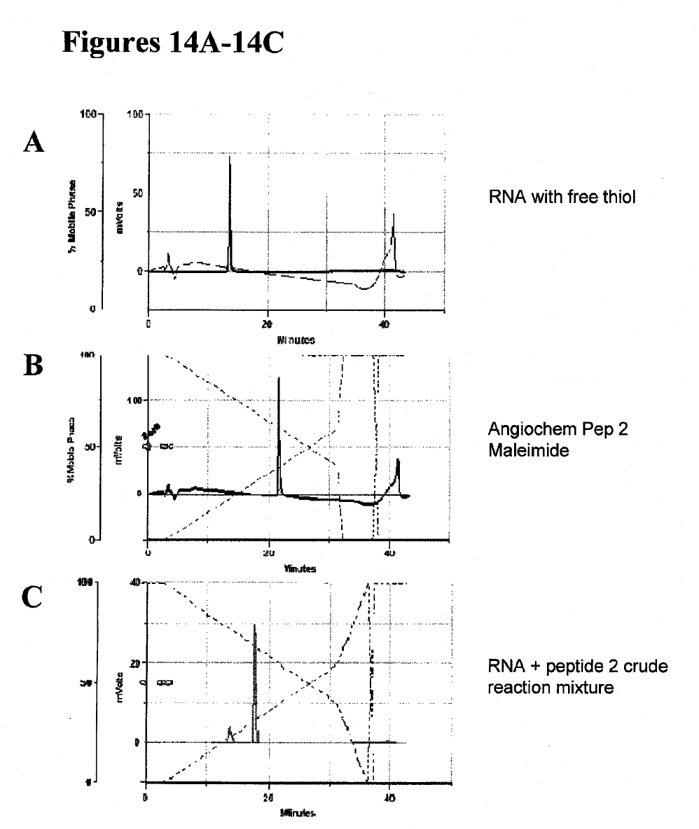

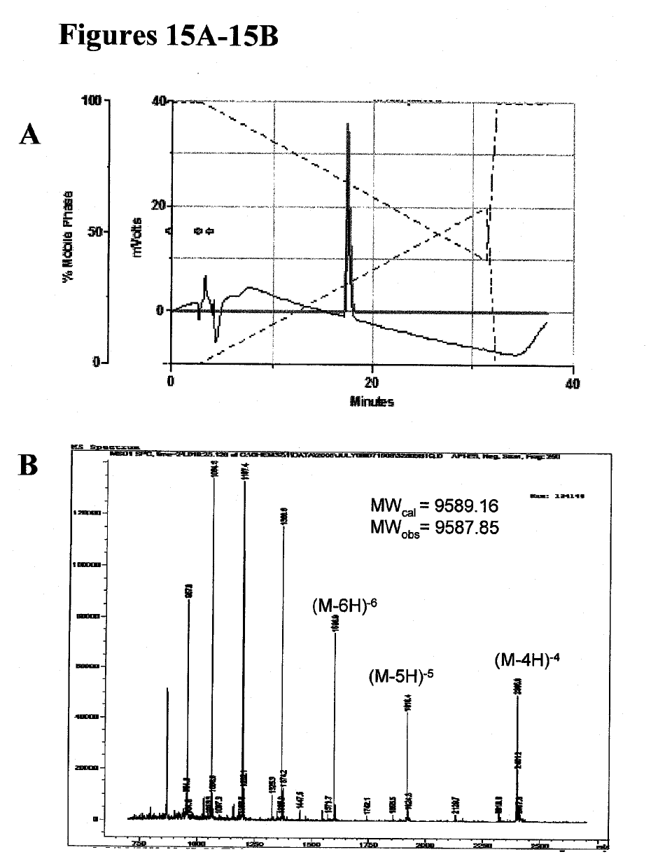

In another exemplary conjugation, the siRNA containing a free thiol was conjugated to Angiopep-2 derivatized with 6-maleimidohexanoic acid (FIG. 13). HPLC traces of the reactants (FIGS. 14A and 14B) and of the reaction mixture (FIG. 14C) indicate a successful reaction. Following further purification, the conjugate was analyzed by HPLC (FIG. 15A) and mass spectroscopy (FIG. 15B), confirmation formation of the conjugate.

### Example 6

### Additional siRNA Conjugates

The siRNA molecules and conjugates shown in Table 4 were also prepared.

TABLE 4
siRNA conjugates

| | | Fluo- |
|---|---|---|
| Name | Description | rescent |
| siRNA-Angiopep-2 | siRNA conjugated on the C-terminal of | No |
| cleavable conjugate | Angiopep-2 (MW 16080) | |
| siRNA-Angiopep-2 | siRNA conjugated on the N-terminal of | No |
| non-cleavable | Angiopep-2 (MW 16172) | |
| conjugate | | |
| Naked siRNA control | Unconjugated siRNA (MW 13475) | No |
| siRNAalexa488- | siRNA conjugated on the C-terminal of | Yes |
| Angiopep-2 cleavable | Angiopep-2. Alexa 488 labeled. (MW | |
| conjugate | 16857) | |
| siRNAalexa488- | siRNA conjugated on the N-terminal of | Yes |
| Angiopep-2 non- | Angiopep-2. Alexa 488 labeled. (MW | |
| cleavable conjugate | 16949) | |
| siRNAalexa488 | siRNA label (MW 14252) | Yes |

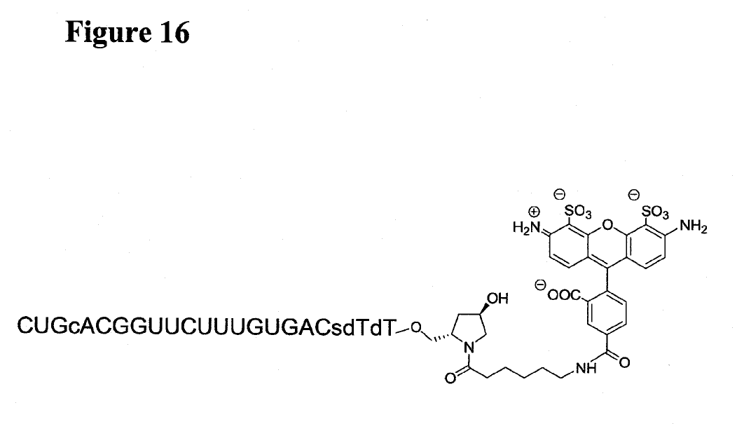

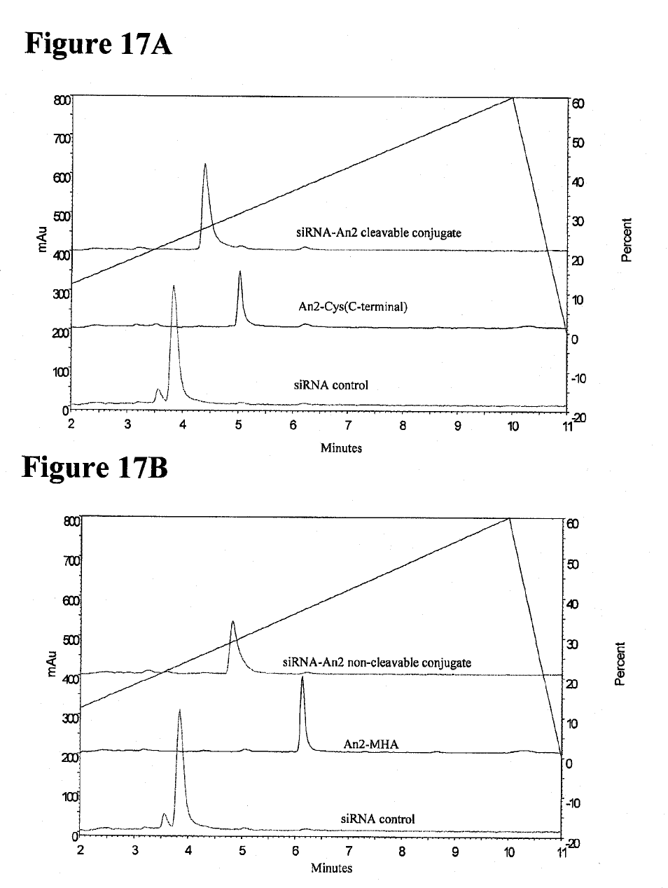

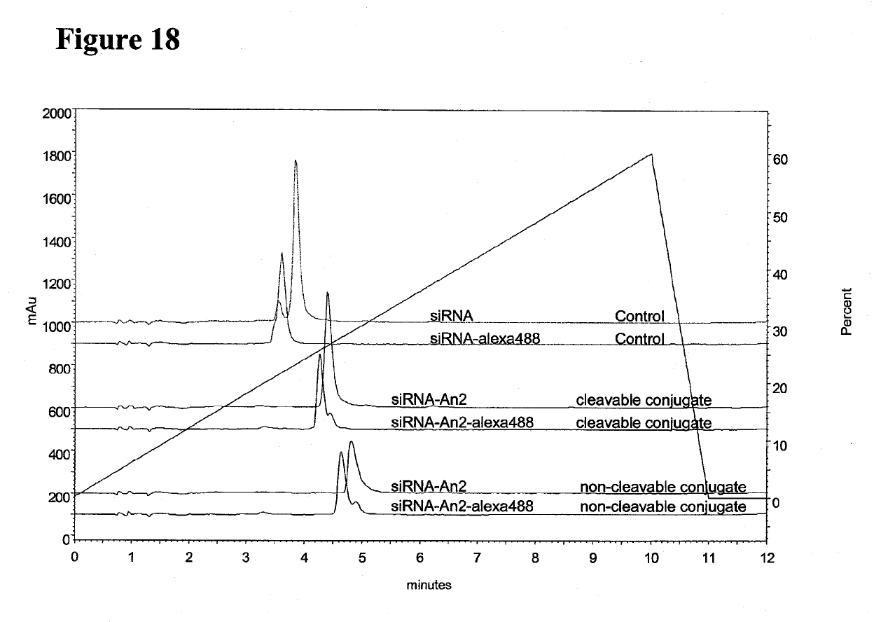

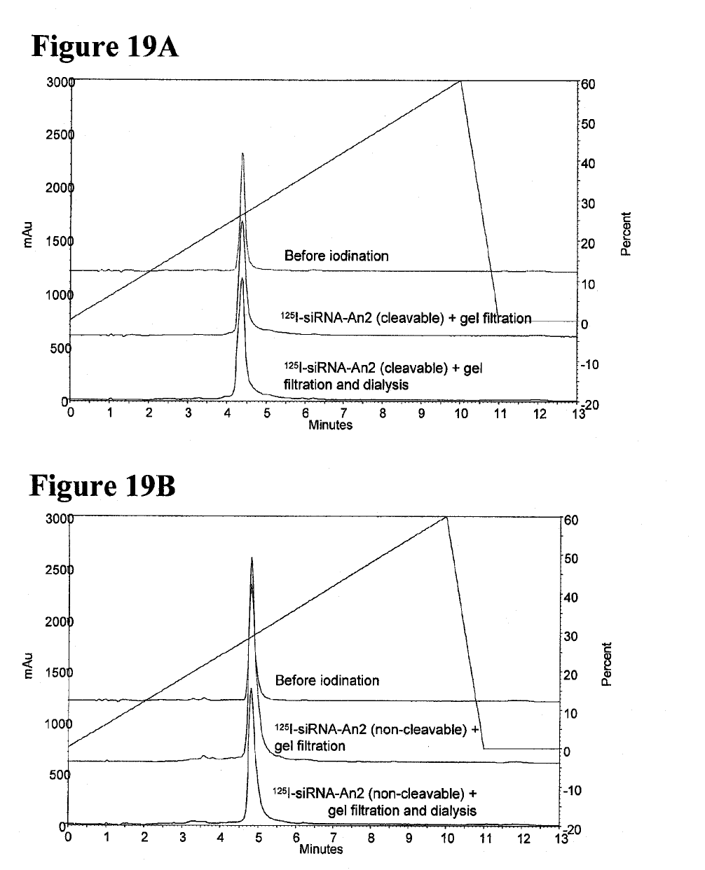

An exemplary RNA-Alexa 488 conjugate is shown in FIG. 16. These molecules described in the table above were analyzed by HPLC on a C18 column using a 50 mM triethylammonium acetate (TEAA), pH 7.0 buffer and acetonitrile gradient. Elution of the cleavable conjugate, Angiopep-2-cys (An2-Cys(C-terminal)), and the siRNA control are shown in FIG. 17A. Similar analysis of the non-cleavable conjugate, Angiopep-MHA, the siRNA control are shown in FIG. 17B. HPLC analysis was also performed on the Alexa 488 labeled conjugates (FIG. 18).

### Example 7

### Iodination of siRNA Conjugates

The siRNA conjugates described in Example 5 were iodinated in phosphobuffered saline (PBS) using Iodobeads. To remove free iodine, the conjugates were separated using gel filtration chromatography on a Sephadex G25 column and subjected to dialysis against PBS using a 10,000 Da molecular weight cutoff. 88% of the radioactivity was associated with the conjugate following gel filtration and 93-95% of the radioactivity was associated with the conjugate following dialysis (data not shown).

To determine whether the conjugates integrity of the conjugates was examined using HPLC. No differences in the HPLC traces of the conjugates before iodination, following iodination and gel filtration, or following iodination, gel filtration, and dialysis were observed for either the cleavable or non-cleavable conjugates. These results indicate that iodination did not affect the integrity of these conjugates.

Specific activity of the iodinated conjugates was also measured, as shown in Table 5 below.

TABLE 5
Specific activity of iodinated siRNA conjugates.

| | |
|---|---|
| siRNA-Angiopep-2 | Specific activity |

| Conjugates | CPM/mg | CPM/mmol |
|---|---|---|
| Cleavable | 1.1 × 10⁸ | 1.8 × 10¹² |
| Non-Cleavable | 1.4 × 10⁸ | 2.3 × 10¹² |
| Angiopep-2 | 5.2 × 10⁸ | 1.2 × 10¹² |

### Example 8

### In Situ Perfusion of siRNA Conjugates

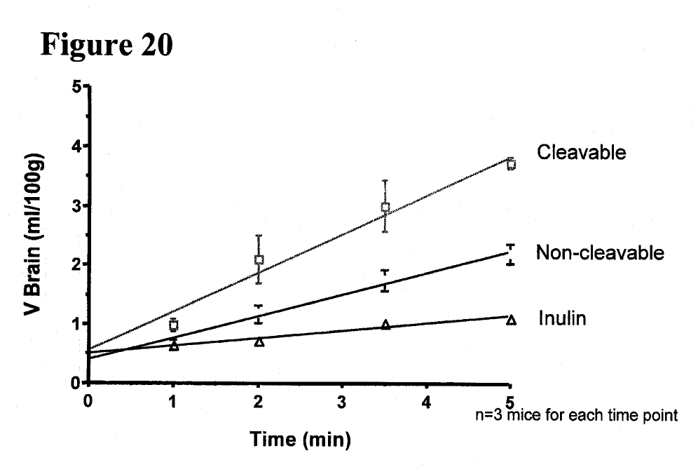

In situ perfusions using 125 nM of the cleavable and non-cleavable conjugates were performed, and uptake into the brain was measured. Inulin was used as a control. Both the cleavable and non-cleavable siRNA conjugates were observed to cross the BBB in the in situ model (FIG. 20). Kᵢₙ values were measured for each protein: The cleavable conjugated has a Kᵢₙ value of 1.1×10⁻⁴ ml/s/g, the non-cleavable has a Kᵢₙ value of 4.7×10⁻⁵ ml/s/g, and inulin has a Kᵢₙ value of 2.1×10⁻⁵ ml/s/g.

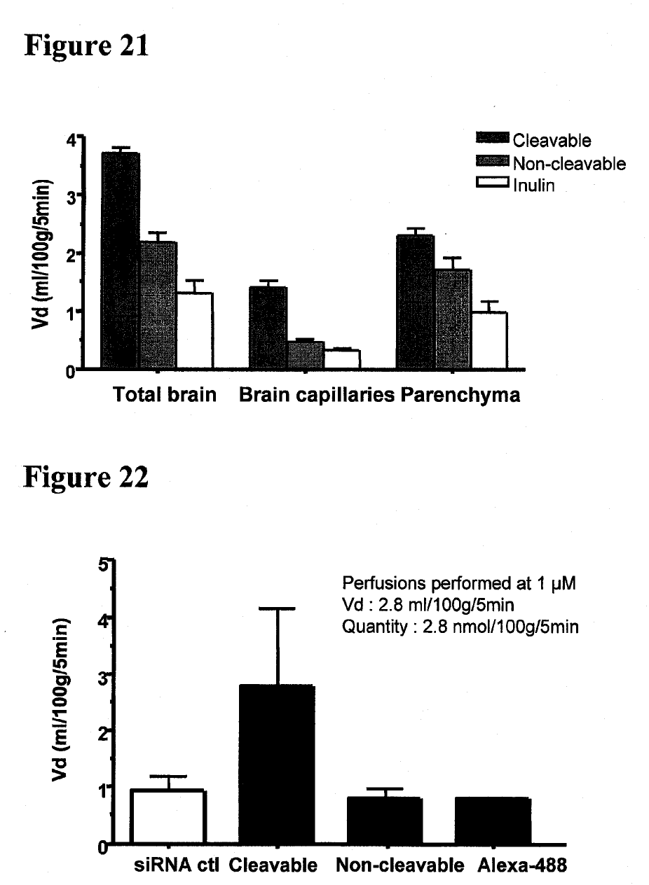

Partition into brain compartments of the siRNA following capillary depletion was also measured. This perfusion was also performed at 125 nM. Greater amounts of both cleavable and non-cleavable siRNA conjugates were observed in total brain, brain capillaries, and in parenchyma, as compared to the inulin control (FIG. 21).

In situ perfusions were also performed using fluorescent siRNA. Cleavable siRNA conjugates exhibited increased perfusion into brain as compared to the control siRNA, Alexa 488, and the non-cleavable siRNA conjugate (FIG. 22). However, high endogenous fluorescence and fluorescence quenching was observed in the experiment.

### Example 9

### Transport of siRNA Conjugates Across the In Vitro BBB Model

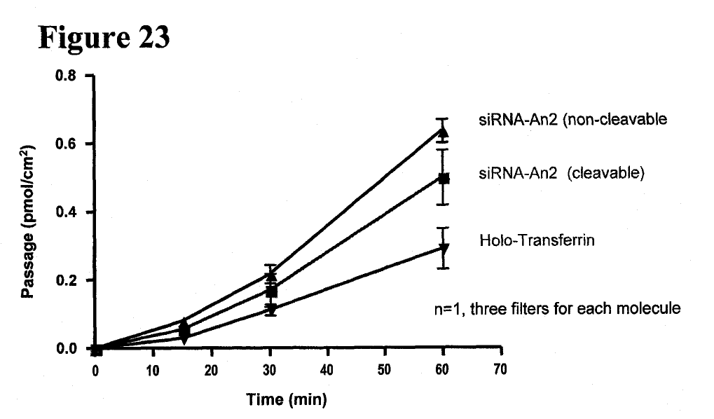

Using an in vitro blood brain barrier model (e.g., as described in U.S. Patent Application Publication 2006/0189515), transport of the siRNA conjugates was measured over time. Holo-transferrin was used as a control. This experiment was performed at 250 nM. TCA precipitation was performed on all fractions, and the amount of radiolabel was measured. Both the non-cleavable and cleavable siRNA conjugates were observed to cross the BBB in vitro more efficiently than holo-transferrin (FIG. 23).

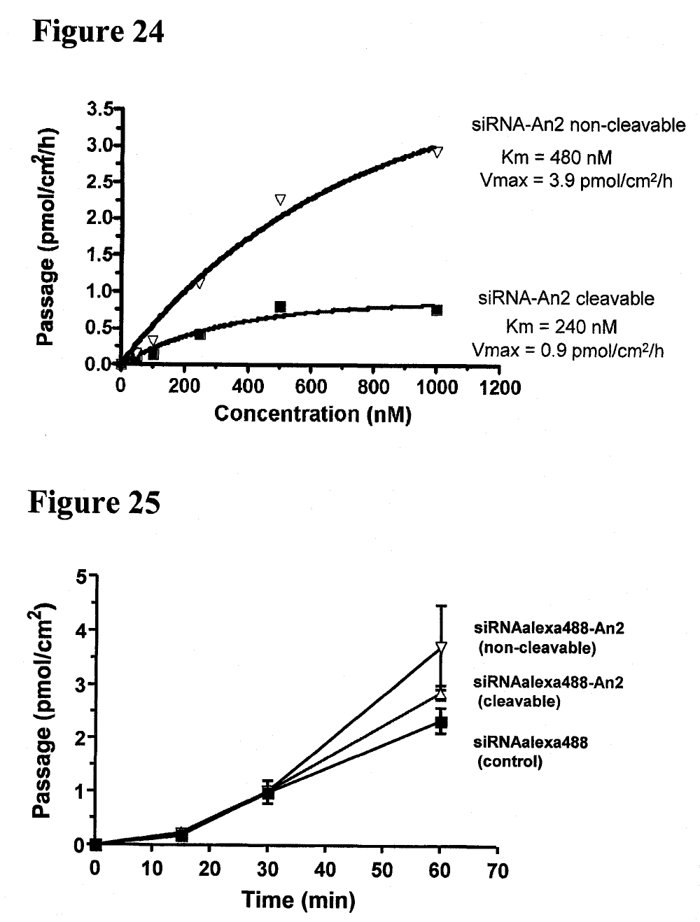

Concentrations of radiolabeled siRNA conjugates between 0 and 1000 nM were tested in the in vitro BBB model and rate of transport was measured (FIG. 24). Based on these data, siRNA transport across the BBB appears to use a saturable mechanism; Kₘ and Vₘₐₓ values for the cleavable and the non-cleavable conjugates were thus calculated. Kₘ and Vₘₐₓ for the non-cleavable siRNA conjugate was measured to be 480 nM and 3.9 pmol/cm²/h, respectively. Kₘ and Vₘₐₓ for the cleavable siRNA conjugate was measured to be 240 nM and 0.9 pmol/cm²/h, respectively.

Transport of fluorescently labeled siRNA conjugates was also measured (FIG. 25). As with the radiolabeled conjugates, the fluorescently labeled conjugates also exhibited increased transport across the BBB, as compared to an unconjugated siRNA control.

### Example 10

### Treatment of Glioblastoma with AngioPep-2/EGFR Conjugates

A human patient diagnosed with glioblastoma is treated with an AngioPep-2/EGFR siRNA conjugate of Example 1. Upon treatment, the conjugate passes through the blood-brain barrier (BBB) and to the cancerous cells in the brain. The presence of siRNA that degrades epidermal growth factor receptor (EGFR) mRNA results in a marked functional silencing of this molecule in the cancerous cells. Treatment results in slower progression or reduced size of the glioblastoma, or complete remission.

### Other Embodiments

All publications, patent applications, and patents mentioned in this specification are herein incorporated by reference, including U.S. Provisional Application Nos. 61/008,880, filed Dec. 20, 2007 and 61/008,825, filed Dec. 20, 2007.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, pharmacology, or related fields are intended to be within the scope of the invention.

What is claimed is:

1. A compound comprising a polypeptide comprising an amino acid sequence having at least 70% sequence identity to any of the sequences set forth in SEQ ID NOS:1-105 and 107-112 conjugated to a nucleic acid molecule.

2. (canceled)

3. The compound of claim 1, wherein said amino acid sequence identity is at least 90%.

4. The compound claim 1, wherein said polypeptide comprises an amino acid sequence set forth in SEQ ID NOS:1-105 and 107-112.

5. (canceled)

6. The compound of claim 4, wherein said polypeptide comprises an amino acid sequence set forth in SEQ ID NO:97.

7. The compound of claim 1, wherein said composition is able to cross a mammalian blood-brain barrier efficiently.

8. The compound of claim 1, wherein said polypeptide is 10 to 50 amino acids in length.

9. The compound of claim 1, wherein said nucleic acid is a ribonucleic acid (RNA) molecule.

10. The compound of claim 1, wherein said nucleic acid is 15 to 25 amino acids in length.

11. The compound of claim 9, wherein said nucleic acid is a short interfering RNA molecule (siRNA), a short hairpin RNA (shRNA) molecule, a double-stranded RNA molecule, or a microRNA (miRNA).

12. The compound of claim 11, wherein said siRNA molecule silences a mammalian epidermal growth factor receptor (EGFR), vascular endothelial growth factor (VEGF), superoxide dismutase 1 (SOD-1), Huntingtin (Htt), α-secretase, β-secretase (BACE), γ-secretase, amyloid precursor protein (APP), sorting nexin-6 (SNX6), LINGO-1, Nogo-A, Nogo receptor 1 (NgR-1), and α-synuclein.

13. The compound of claim 11, wherein said siRNA molecule silences a mammalian epidermal growth factor receptor (EGFR).

14. The compound of claim 11, wherein said siRNA molecule comprises a nucleotide sequence comprising at least 80% sequence identity to any of the sequences set forth in SEQ ID NOS:117-119.

15. The compound of claim 11, wherein said siRNA molecule comprises a nucleotide sequence comprising any of the sequences set forth in SEQ ID NOS:117-119.

16-30. (canceled)

31. The compound of claim 1, wherein said compound is purified.

32. The compound of claim 1, wherein said polypeptide is produced by recombinant genetic technology or by chemical synthesis.

33. (canceled)

34. A composition comprising the compound of claim 1 and a pharmaceutically acceptable carrier.

35-39. (canceled)

40. A method of treating a subject having a neurodegenerative disease comprising providing to said subject the compound of claim 1 in a therapeutically effective amount.

41. The method of claim 40, wherein said neurodegenerative disease is multiple sclerosis, schizophrenia, epilepsy, Alzheimer's disease, Parkinson's disease, Huntington's disease, amyotrophic lateral sclerosis (ALS), or a stroke.

42. A method of treating a mammal having a lysosomal storage disease comprising providing to said mammal the compound of claim 1 in a therapeutically effective amount.

43. The method of claim 42, wherein said lysosomal storage disease is mucopolysaccharidosis (MPS-I; i.e., Hurler syndrome, Scheie syndrome), MPS-II (Hunter syndrome), MPS-IIIA (Sanfilippo syndrome A), MPS-IIIB (Sanfilippo syndrome B), MPS-IIIC (Sanfilippo syndrome C), MPS-IIID (Sanfilippo syndrome D), MPS-VII (Sly syndrome), Gaucher's disease, Niemann-Pick disease, Fabry disease, Farber's disease, Wolman's disease, Tay-Sachs disease, Sandhoff disease, metachromatic leukodystrophy, or Krabbé disease.

44. A method of treating a mammal with a cancer comprising providing to said mammal the compound of claim 1 in a therapeutically effective amount.

45. The method of claim 44, wherein said cancer is in the brain or central nervous system (CNS).

46. The method of claim 44, wherein said cancer is a brain tumor, a brain tumor metastasis, or a tumor that has metastasized to the brain.

47. The method of claim 44, wherein said cancer is a glioma a glioblastoma, hepatocellular carcinoma, or lung cancer.

48-49. (canceled)

50. A method of synthesizing the compound of claim 1 comprising conjugating a polypeptide comprising an amino acid sequence comprising at least 80% sequence identity to any of the sequences set forth in SEQ ID NOs:1-105 and 107-112 to a nucleic acid.

51. The method of claim 50, wherein said conjugating comprises a covalent bond.

52. The method of claim 51, wherein said covalent bond is a disulfide bond.


SEQUENCE LISTING (121 sequences)

SEQ ID NO: 1 - protein, 19 aa, Artificial Sequence
  Description of Artificial Sequence Synthetic peptide
TFVYGGCRAKRNNFKSAED

SEQ ID NO: 2 - protein, 19 aa, Artificial Sequence
  Description of Artificial Sequence Synthetic peptide
TFQYGGCMGNGNNFVTEKE

SEQ ID NO: 3 - protein, 19 aa, Artificial Sequence
  Description of Artificial Sequence Synthetic peptide
PFFYGGCGGNRNNFDTEEY

SEQ ID NO: 4 - protein, 19 aa, Artificial Sequence
  Description of Artificial Sequence Synthetic peptide
SFYYGGCLGNKNNYLREEE

SEQ ID NO: 5 - protein, 19 aa, Artificial Sequence
  Description of Artificial Sequence Synthetic peptide
TFFYGGCRAKRNNFKRAKY

SEQ ID NO: 6 - protein, 19 aa, Artificial Sequence
  Description of Artificial Sequence Synthetic peptide
TFFYGGCRGKRNNFKRAKY

SEQ ID NO: 7 - protein, 19 aa, Artificial Sequence
  Description of Artificial Sequence Synthetic peptide
TFFYGGCRAKKNNYKRAKY

SEQ ID NO: 8 - protein, 19 aa, Artificial Sequence
  Description of Artificial Sequence Synthetic peptide
TFFYGGCRGKKNNFKRAKY

SEQ ID NO: 9 - protein, 19 aa, Artificial Sequence
  Description of Artificial Sequence Synthetic peptide
TFQYGGCRAKRNNFKRAKY

SEQ ID NO: 10 - protein, 19 aa, Artificial Sequence
  Description of Artificial Sequence Synthetic peptide
TFQYGGCRGKKNNFKRAKY

SEQ ID NO: 11 - protein, 19 aa, Artificial Sequence
  Description of Artificial Sequence Synthetic peptide
TFFYGGCLGKRNNFKRAKY

SEQ ID NO: 12 - protein, 19 aa, Artificial Sequence
  Description of Artificial Sequence Synthetic peptide
TFFYGGSLGKRNNFKRAKY

SEQ ID NO: 13 - protein, 19 aa, Artificial Sequence
  Description of Artificial Sequence Synthetic peptide
PFFYGGCGGKKNNFKRAKY

SEQ ID NO: 14 - protein, 19 aa, Artificial Sequence
  Description of Artificial Sequence Synthetic peptide
TFFYGGCRGKGNNYKRAKY

SEQ ID NO: 15 - protein, 19 aa, Artificial Sequence
  Description of Artificial Sequence Synthetic peptide
PFFYGGCRGKRNNFLRAKY

SEQ ID NO: 16 - protein, 19 aa, Artificial Sequence
  Description of Artificial Sequence Synthetic peptide
TFFYGGCRGKRNNFKREKY

SEQ ID NO: 17 - protein, 19 aa, Artificial Sequence
  Description of Artificial Sequence Synthetic peptide
PFFYGGCRAKKNNFKRAKE

SEQ ID NO: 18 - protein, 19 aa, Artificial Sequence
  Description of Artificial Sequence Synthetic peptide
TFFYGGCRGKRNNFKRAKD

SEQ ID NO: 19 - protein, 19 aa, Artificial Sequence
  Description of Artificial Sequence Synthetic peptide
TFFYGGCRAKRNNFDRAKY

SEQ ID NO: 20 - protein, 19 aa, Artificial Sequence
  Description of Artificial Sequence Synthetic peptide
TFFYGGCRGKKNNFKRAEY

SEQ ID NO: 21 - protein, 19 aa, Artificial Sequence
  Description of Artificial Sequence Synthetic peptide
PFFYGGCGANRNNFKRAKY

SEQ ID NO: 22 - protein, 19 aa, Artificial Sequence
  Description of Artificial Sequence Synthetic peptide
TFFYGGCGGKKNNFKTAKY

SEQ ID NO: 23 - protein, 19 aa, Artificial Sequence
  Description of Artificial Sequence Synthetic peptide
TFFYGGCRGNRNNFLRAKY

SEQ ID NO: 24 - protein, 19 aa, Artificial Sequence
  Description of Artificial Sequence Synthetic peptide
TFFYGGCRGNRNNFKTAKY

SEQ ID NO: 25 - protein, 19 aa, Artificial Sequence
  Description of Artificial Sequence Synthetic peptide
TFFYGGSRGNRNNFKTAKY

SEQ ID NO: 26 - protein, 19 aa, Artificial Sequence
  Description of Artificial Sequence Synthetic peptide
TFFYGGCLGNGNNFKRAKY

SEQ ID NO: 27 - protein, 19 aa, Artificial Sequence
  Description of Artificial Sequence Synthetic peptide
TFFYGGCLGNRNNFLRAKY

SEQ ID NO: 28 - protein, 19 aa, Artificial Sequence
  Description of Artificial Sequence Synthetic peptide
TFFYGGCLGNRNNFKTAKY

SEQ ID NO: 29 - protein, 19 aa, Artificial Sequence
  Description of Artificial Sequence Synthetic peptide
TFFYGGCRGNGNNFKSAKY

SEQ ID NO: 30 - protein, 19 aa, Artificial Sequence
  Description of Artificial Sequence Synthetic peptide
TFFYGGCRGKKNNFDREKY

SEQ ID NO: 31 - protein, 19 aa, Artificial Sequence
  Description of Artificial Sequence Synthetic peptide
TFFYGGCRGKRNNFLREKE

SEQ ID NO: 32 - protein, 19 aa, Artificial Sequence
  Description of Artificial Sequence Synthetic peptide
TFFYGGCRGKGNNFDRAKY

SEQ ID NO: 33 - protein, 19 aa, Artificial Sequence
  Description of Artificial Sequence Synthetic peptide
TFFYGGSRGKGNNFDRAKY

SEQ ID NO: 34 - protein, 19 aa, Artificial Sequence
  Description of Artificial Sequence Synthetic peptide
TFFYGGCRGNGNNFVTAKY

SEQ ID NO: 35 - protein, 19 aa, Artificial Sequence
  Description of Artificial Sequence Synthetic peptide
PFFYGGCGGKGNNYVTAKY

SEQ ID NO: 36 - protein, 19 aa, Artificial Sequence
  Description of Artificial Sequence Synthetic peptide
TFFYGGCLGKGNNFLTAKY

SEQ ID NO: 37 - protein, 19 aa, Artificial Sequence
  Description of Artificial Sequence Synthetic peptide
SFFYGGCLGNKNNFLTAKY

SEQ ID NO: 38 - protein, 19 aa, Artificial Sequence
  Description of Artificial Sequence Synthetic peptide
TFFYGGCGGNKNNFVREKY

SEQ ID NO: 39 - protein, 19 aa, Artificial Sequence
  Description of Artificial Sequence Synthetic peptide
TFFYGGCMGNKNNFVREKY

SEQ ID NO: 40 - protein, 19 aa, Artificial Sequence
  Description of Artificial Sequence Synthetic peptide
TFFYGGSMGNKNNFVREKY

SEQ ID NO: 41 - protein, 19 aa, Artificial Sequence
  Description of Artificial Sequence Synthetic peptide
PFFYGGCLGNRNNYVREKY

SEQ ID NO: 42 - protein, 19 aa, Artificial Sequence
  Description of Artificial Sequence Synthetic peptide
TFFYGGCLGNRNNFVREKY

SEQ ID NO: 43 - protein, 19 aa, Artificial Sequence
  Description of Artificial Sequence Synthetic peptide
TFFYGGCLGNKNNYVREKY

SEQ ID NO: 44 - protein, 19 aa, Artificial Sequence
  Description of Artificial Sequence Synthetic peptide
TFFYGGCGGNGNNFLTAKY

SEQ ID NO: 45 - protein, 19 aa, Artificial Sequence
  Description of Artificial Sequence Synthetic peptide
TFFYGGCRGNRNNFLTAEY

SEQ ID NO: 46 - protein, 19 aa, Artificial Sequence
  Description of Artificial Sequence Synthetic peptide
TFFYGGCRGNGNNFKSAEY

SEQ ID NO: 47 - protein, 19 aa, Artificial Sequence
  Description of Artificial Sequence Synthetic peptide
PFFYGGCLGNKNNFKTAEY

SEQ ID NO: 48 - protein, 19 aa, Artificial Sequence
  Description of Artificial Sequence Synthetic peptide
TFFYGGCRGNRNNFKTEEY

SEQ ID NO: 49 - protein, 19 aa, Artificial Sequence
  Description of Artificial Sequence Synthetic peptide
TFFYGGCRGKRNNFKTEED

SEQ ID NO: 50 - protein, 19 aa, Artificial Sequence
  Description of Artificial Sequence Synthetic peptide
PFFYGGCGGNGNNFVREKY

SEQ ID NO: 51 - protein, 19 aa, Artificial Sequence
  Description of Artificial Sequence Synthetic peptide
SFFYGGCMGNGNNFVREKY

SEQ ID NO: 52 - protein, 19 aa, Artificial Sequence
  Description of Artificial Sequence Synthetic peptide
PFFYGGCGGNGNNFLREKY

SEQ ID NO: 53 - protein, 19 aa, Artificial Sequence
  Description of Artificial Sequence Synthetic peptide
TFFYGGCLGNGNNFVREKY

SEQ ID NO: 54 - protein, 19 aa, Artificial Sequence
  Description of Artificial Sequence Synthetic peptide
SFFYGGCLGNGNNYLREKY

SEQ ID NO: 55 - protein, 19 aa, Artificial Sequence
  Description of Artificial Sequence Synthetic peptide
TFFYGGSLGNGNNFVREKY

SEQ ID NO: 56 - protein, 19 aa, Artificial Sequence
  Description of Artificial Sequence Synthetic peptide
TFFYGGCRGNGNNFVTAEY

SEQ ID NO: 57 - protein, 19 aa, Artificial Sequence
  Description of Artificial Sequence Synthetic peptide
TFFYGGCLGKGNNFVSAEY

SEQ ID NO: 58 - protein, 19 aa, Artificial Sequence
  Description of Artificial Sequence Synthetic peptide
TFFYGGCLGNRNNFDRAEY

SEQ ID NO: 59 - protein, 19 aa, Artificial Sequence
  Description of Artificial Sequence Synthetic peptide
TFFYGGCLGNRNNFLREEY

SEQ ID NO: 60 - protein, 19 aa, Artificial Sequence
  Description of Artificial Sequence Synthetic peptide
TFFYGGCLGNKNNYLREEY

SEQ ID NO: 61 - protein, 19 aa, Artificial Sequence
  Description of Artificial Sequence Synthetic peptide
PFFYGGCGGNRNNYLREEY

SEQ ID NO: 62 - protein, 19 aa, Artificial Sequence
  Description of Artificial Sequence Synthetic peptide
PFFYGGSGGNRNNYLREEY

SEQ ID NO: 63 - protein, 19 aa, Artificial Sequence
  Description of Artificial Sequence Synthetic peptide
MRPDFCLEPPYTGPCVARI

SEQ ID NO: 64 - protein, 21 aa, Artificial Sequence
  Description of Artificial Sequence Synthetic peptide
ARIIRYFYNAKAGLCQTFVYG

SEQ ID NO: 65 - protein, 22 aa, Artificial Sequence
  Description of Artificial Sequence Synthetic peptide
YGGCRAKRNNYKSAEDCMRTCG

SEQ ID NO: 66 - protein, 22 aa, Artificial Sequence
  Description of Artificial Sequence Synthetic peptide
PDFCLEPPYTGPCVARIIRYFY

SEQ ID NO: 67 - protein, 19 aa, Artificial Sequence
  Description of Artificial Sequence Synthetic peptide
TFFYGGCRGKRNNFKTEEY

SEQ ID NO: 68 - protein, 19 aa, Artificial Sequence
  Description of Artificial Sequence Synthetic peptide
KFFYGGCRGKRNNFKTEEY

SEQ ID NO: 69 - protein, 19 aa, Artificial Sequence
  Description of Artificial Sequence Synthetic peptide
TFYYGGCRGKRNNYKTEEY

SEQ ID NO: 70 - protein, 19 aa, Artificial Sequence
  Description of Artificial Sequence Synthetic peptide
TFFYGGSRGKRNNFKTEEY

SEQ ID NO: 71 - protein, 20 aa, Artificial Sequence
  Description of Artificial Sequence Synthetic peptide
CTFFYGCCRGKRNNFKTEEY

SEQ ID NO: 72 - protein, 20 aa, Artificial Sequence
  Description of Artificial Sequence Synthetic peptide
TFFYGGCRGKRNNFKTEEYC

SEQ ID NO: 73 - protein, 20 aa, Artificial Sequence
  Description of Artificial Sequence Synthetic peptide
CTFFYGSCRGKRNNFKTEEY

SEQ ID NO: 74 - protein, 20 aa, Artificial Sequence
  Description of Artificial Sequence Synthetic peptide
TFFYGGSRGKRNNFKTEEYC

SEQ ID NO: 75 - protein, 19 aa, Artificial Sequence
  Description of Artificial Sequence Synthetic peptide
PFFYGGCRGKRNNFKTEEY

SEQ ID NO: 76 - protein, 19 aa, Artificial Sequence
  Description of Artificial Sequence Synthetic peptide
TFFYGGCRGKRNNFKTKEY

SEQ ID NO: 77 - protein, 19 aa, Artificial Sequence
  Description of Artificial Sequence Synthetic peptide
TFFYGGKRGKRNNFKTEEY

SEQ ID NO: 78 - protein, 19 aa, Artificial Sequence
  Description of Artificial Sequence Synthetic peptide
TFFYGGCRGKRNNFKTKRY

SEQ ID NO: 79 - protein, 19 aa, Artificial Sequence
  Description of Artificial Sequence Synthetic peptide
TFFYGGKRGKRNNFKTAEY

SEQ ID NO: 80 - protein, 19 aa, Artificial Sequence
  Description of Artificial Sequence Synthetic peptide
TFFYGGKRGKRNNFKTAGY

SEQ ID NO: 81 - protein, 19 aa, Artificial Sequence
  Description of Artificial Sequence Synthetic peptide
TFFYGGKRGKRNNFKREKY

SEQ ID NO: 82 - protein, 19 aa, Artificial Sequence
  Description of Artificial Sequence Synthetic peptide
TFFYGGKRGKRNNFKRAKY

SEQ ID NO: 83 - protein, 19 aa, Artificial Sequence
  Description of Artificial Sequence Synthetic peptide
TFFYGGCLGNRNNFKTEEY

SEQ ID NO: 84 - protein, 19 aa, Artificial Sequence
  Description of Artificial Sequence Synthetic peptide
TFFYGCGRGKRNNFKTEEY

SEQ ID NO: 85 - protein, 19 aa, Artificial Sequence
  Description of Artificial Sequence Synthetic peptide
TFFYGGRCGKRNNFKTEEY

SEQ ID NO: 86 - protein, 19 aa, Artificial Sequence
  Description of Artificial Sequence Synthetic peptide
TFFYGGCLGNGNNFDTEEE

SEQ ID NO: 87 - protein, 19 aa, Artificial Sequence
  Description of Artificial Sequence Synthetic peptide
TFQYGGCRGKRNNFKTEEY

SEQ ID NO: 88 - protein, 19 aa, Artificial Sequence
  Description of Artificial Sequence Synthetic peptide
YNKEFGTFNTKGCERGYRF

SEQ ID NO: 89 - protein, 19 aa, Artificial Sequence
  Description of Artificial Sequence Synthetic peptide
RFKYGGCLGNMNNFETLEE

SEQ ID NO: 90 - protein, 19 aa, Artificial Sequence
  Description of Artificial Sequence Synthetic peptide
RFKYGGCLGNKNNFLRLKY

SEQ ID NO: 91 - protein, 19 aa, Artificial Sequence
  Description of Artificial Sequence Synthetic peptide
RFKYGGCLGNKNNYLRLKY

SEQ ID NO: 92 - protein, 22 aa, Artificial Sequence
  Description of Artificial Sequence Synthetic peptide
KTKRKRKKQRVKIAYEEIFKNY

SEQ ID NO: 93 - protein, 15 aa, Artificial Sequence
  Description of Artificial Sequence Synthetic peptide
KTKRKRKKQRVKIAY

SEQ ID NO: 94 - protein, 17 aa, Artificial Sequence
  Description of Artificial Sequence Synthetic peptide
RGGRLSYSRRFSTSTGR

SEQ ID NO: 95 - protein, 10 aa, Artificial Sequence
  Description of Artificial Sequence Synthetic peptide
RRLSYSRRRF

SEQ ID NO: 96 - protein, 16 aa, Artificial Sequence
  Description of Artificial Sequence Synthetic peptide
RQIKIWFQNRRMKWKK

SEQ ID NO: 97 - protein, 19 aa, Artificial Sequence
  Description of Artificial Sequence Synthetic peptide
TFFYGGSRGKRNNFKTEEY

SEQ ID NO: 98 - protein, 59 aa, Artificial Sequence
  Description of Artificial Sequence Synthetic polypeptide
MRPDFCLEPPYTGPCVARIIRYFYNAKAGLCQTFVYGGCRAKRNNFKSAEDCMRTCGGA

SEQ ID NO: 99 - protein, 19 aa, Artificial Sequence
  Description of Artificial Sequence Synthetic peptide
TFFYGGCRGKRNNFKTKEY

SEQ ID NO: 100 - protein, 19 aa, Artificial Sequence
  Description of Artificial Sequence Synthetic peptide
RFKYGGCLGNKNNYLRLKY

SEQ ID NO: 101 - protein, 19 aa, Artificial Sequence
  Description of Artificial Sequence Synthetic peptide
TFFYGGCRAKRNNFKRAKY

SEQ ID NO: 102 - protein, 35 aa, Artificial Sequence
  Description of Artificial Sequence Synthetic polypeptide
NAKAGLCQTFVYGGCLAKRNNFESAEDCMRTCGGA

SEQ ID NO: 103 - protein, 24 aa, Artificial Sequence
  Description of Artificial Sequence Synthetic peptide
YGGCRAKRNNFKSAEDCMRTCGGA

SEQ ID NO: 104 - protein, 22 aa, Artificial Sequence
  Description of Artificial Sequence Synthetic peptide
GLCQTFVYGGCRAKRNNFKSAE

SEQ ID NO: 105 - protein, 20 aa, Artificial Sequence
  Description of Artificial Sequence Synthetic peptide
LCQTFVYGGCEAKRNNFKSA

SEQ ID NO: 106 - DNA, 180 bp, Artificial Sequence
  Description of Artificial Sequence Synthetic construct
atgagaccagatttctgcctcgagccgccgtacactgggccctgcaaagctcgtatcatccgttacttctacaatgcaaaggcaggcctgtgtcagaccttcgtatacggcggctgcagagctaagcgtaacaacttcaaatccgcggaagactgcatgcgtacttgcggtggtgcttag

SEQ ID NO: 107 - protein, 19 aa, Artificial Sequence
  Description of Artificial Sequence Synthetic peptide
TFFYGGSRGKRNNFKTEEY

SEQ ID NO: 108 - protein, 19 aa, Artificial Sequence
  Description of Artificial Sequence Synthetic peptide
RFFYGGSRGKRNNFKTEEY

SEQ ID NO: 109 - protein, 19 aa, Artificial Sequence
  Description of Artificial Sequence Synthetic peptide
RFFYGGSRGKRNNFKTEEY

SEQ ID NO: 110 - protein, 19 aa, Artificial Sequence
  Description of Artificial Sequence Synthetic peptide
RFFYGGSRGKRNNFRTEEY

SEQ ID NO: 111 - protein, 19 aa, Artificial Sequence
  Description of Artificial Sequence Synthetic peptide
TFFYGGSRGKRNNFRTEEY

SEQ ID NO: 112 - protein, 19 aa, Artificial Sequence
  Description of Artificial Sequence Synthetic peptide
TFFYGGSRGRRNNFRTEEY

SEQ ID NO: 113 - protein, 20 aa, Artificial Sequence
  Description of Artificial Sequence Synthetic peptide
CTFFYGGSRGKRNNFKTEEY

SEQ ID NO: 114 - protein, 20 aa, Artificial Sequence
  Description of Artificial Sequence Synthetic peptide
TFFYGGSRGKRNNFKTEEYC

SEQ ID NO: 115 - protein, 20 aa, Artificial Sequence
  Description of Artificial Sequence Synthetic peptide
CTFFYGGSRGRRNNFRTEEY

SEQ ID NO: 116 - protein, 20 aa, Artificial Sequence
  Description of Artificial Sequence Synthetic peptide
TFFYGGSRGRRNNFRTEEYC

SEQ ID NO: 117 - RNA, 19 nt, Artificial Sequence
  Description of Artificial Sequence Synthetic construct
ggagcugcccaugagaaau

SEQ ID NO: 118 - RNA, 18 nt, Artificial Sequence
  Description of Artificial Sequence Synthetic construct
auuucucaugggcagcuc

SEQ ID NO: 119 - DNA, 19 bp, Artificial Sequence
  Description of Artificial Sequence Synthetic construct
ggagtaccctgatgagatc

SEQ ID NO: 120 - DNA, 21 bp, Artificial Sequence
  Description of Artificial Sequence Synthetic construct
gucacaaagaaccgugcagtt

SEQ ID NO: 121 - DNA, 21 bp, Artificial Sequence
  Description of Artificial Sequence Synthetic construct
cugcacgguucuuugugactt